(19) United States
(12) Patent Application Publication
(10) Pub. No.: US 2008/0188936 A1
(43) Pub. Date: Aug. 7, 2008

(54) SYSTEM AND METHOD FOR REPAIRING TENDONS AND LIGAMENTS

(75) Inventors: Robert J. Ball, West Olive, MI (US); Thomas D. Egan, Marblehead, MA (US); Paul V. Fenton, Marblehead, MA (US); Kevin L. Ohashi, Jamaica Plain, MA (US); Dale R. Peterson, La Jolla, CA (US); Peter Sorenson, Salem, MA (US); Douglas Snell, Amesbury, MA (US); John Yannone, Seabrook, NH (US); Patrick Fenton, Marblehead, MA (US)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(73) Assignee: Tornier, Inc., Edina, MN (US)

(21) Appl. No.: 12/025,449

(22) Filed: Feb. 4, 2008

Related U.S. Application Data
(60) Provisional application No. 60/899,099, filed on Feb. 2, 2007.
(60) Provisional application No. 60/900,402, filed on Feb. 9, 2007.
(60) Provisional application No. 60/900,403, filed on Feb. 9, 2007.

(51) Int. Cl. A61F 2/08

(52) U.S. Cl. 623/13.14; 623/13.11; 623/13.13

(57) ABSTRACT

An implant and method for the repair of a tendon or a ligament along at least one load direction. The implant includes at least one first anchor portion and at least one tension member oriented along a load direction. The first anchor portion preferably has a larger surface area of engagement with the tendon or ligament to spread loads across more tissue. The tension member is preferably secured to the first anchor portion with an overlapping attachment. Tension on the tension member is preferably adjustable by the surgeon.

The present application claims the benefit of U.S. Provisional Application Ser. Nos. 60/899,099, entitled Ligament and Tendon-to-Bone Repair Augmentation Device, filed Feb. 2, 2007, 60/900,402, entitled Thermally Welded Fabric Assembly and Method, filed Feb. 9, 2007 and 60/900,403, entitled Fabric-to-Bone Thermal Weld System and Method, filed Feb. 9, 2007, the complete disclosures of which are hereby incorporated by reference.

### FIELD OF THE INVENTION

The present invention relates to surgical repair of torn tendons and ligaments in an animal, and in particular, to open and arthroscopic orthopedic surgical repair of torn tendons and ligaments in the body, such as arthroscopic repair of torn rotator cuff tissue in the human shoulder.

### BACKGROUND OF THE INVENTION

As illustrated in FIG. 1, the rotator cuff 20 is the complex of four muscles that arise from the scapula 22 and whose tendons blend in with the subjacent capsule as they attach to the tuberosities of the humerus 24. The subscapularis 26 arises from the anterior aspect of the scapula 20 and attaches over much of the lesser tuberosity. The supraspinatus muscle 28 arises from the supraspinatus fossa of the posterior scapula, passes beneath the acromion and the acromioclavicular joint, and attaches to the superior aspect of the greater tuberosity 30. The infraspinatus muscle 32 arises from the infraspinous fossa of the posterior scapula and attaches to the posterolateral aspect of the greater tuberosity 30. The teres minor 34 arises from the lower lateral aspect of the scapula 20 and attaches to the lower aspect of the greater tuberosity 30. Proper functioning of the rotator, 3 to 4 millimeters thick, depends on the fundamental centering and stabilizing role of the humeral head 31 with respect to sliding action during anterior and lateral lifting and rotation movements of the arm.

The insertion of these tendons as a continuous cuff 20 around the humeral head 31 permits the cuff muscles to provide an infinite variety of moments to rotate the humerus 24 and to oppose unwanted components of the deltoid and pectoralis muscle forces. The insertion of the infraspinatus 32 overlaps that of the supraspinatus 28 to some extent. Each of the other tendons 26, 34 also interlaces its fibers to some extent with its neighbor's tendons. The tendons splay out and interdigitat to form a common continuous insertion on the humerus 24. The biceps tendon is ensheathed by interwoven fibers derived from the subscapularis and supraspinatus.

The mechanics of the rotator cuff 20 is complex. The cuff muscles 20 rotate the humerus 24 with respect to the scapula 22, compress the humeral head 31 into the glenoid fossa providing a critical stabilizing mechanism to the shoulder (known as concavity compression), and provide muscular balance. The supraspinatus and infraspinatus provide 45 percent of abduction and 90 percent of external rotation strength. The supraspinatus and deltoid muscles are equally responsible for producing torque about the shoulder joint in the functional planes of motion.

The rotator cuff muscles 20 are critical elements of this shoulder muscle balance equation. The human shoulder has no fixed axis. In a specified position, activation of a muscle creates a unique set of rotational moments. For example, the anterior deltoid can exert moments in forward elevation, internal rotation, and cross-body movement. If forward elevation is to occur without rotation, the cross-body and internal rotation moments of this muscle must be neutralized by other muscles, such as the posterior deltoid and infraspinatus. As another example, use of the latissimus dorsi in a movement of pure internal rotation requires that its adduction moment by neutralized by the superior cuff and deltoid. Conversely, use of the latissimus in a movement of pure adduction requires that its internal rotation moment be neutralized by the posterior cuff and posterior deltoid muscles.

The timing and magnitude of these balancing muscle effects must be precisely coordinated to avoid unwanted directions of humeral motion. Thus the simplified view of muscles as isolated motors, or as members of force couples must give way to an understanding that all shoulder muscles function together in a precisely coordinated way—opposing muscles canceling out undesired elements leaving only the net torque necessary to produce the desired action.

By contrast, muscles in the knee generate torques primarily about a single axis of flexion-extension. If the quadriceps pull is a bit off-center, the knee still extends. Consequently, the human shoulder is a good tool to illustrate the present method and apparatus.

The suprasinatus 28 frequently tears away from the humerus 24 due to high stress activity or traumatic injury. FIG. 2 is an anterior view of a human left shoulder with a torn supraspinatus tendon 28. FIG. 3 is a posterior view of a human right shoulder with a torn supraspinatus tendon 28. The supraspinatus 28 has separated from the humerus 24 along its lateral edge 36 away from its attachment surface or “footprint” in the greater tuberosity 30.

Surgical repair is usually accomplished by reattaching the tendon back in apposition to the region of bone from which it tore. For the supraspinatus tendon 28 this attachment region, commonly called the “footprint”, occurs in a feature of the humerus 24 called the greater tuberosity 30. Repair is generally accomplished by sutured fixation the tendon 28 directly to holes or tunnels created in the bone, or to anchoring devices embedded in the bone surface.

FIG. 4 shows a conventional arthroscopic repair of the torn suprasinatus tendon 28. The margins of the tear have been brought together at a convergence line 50 and closed by tendon-to-tendon stitches 52. The lateral edge 54 has been brought into apposition with the greater tuberosity 30 and secured in place through the use of four sutures 56 secured to two bone anchors 58 driven into the bone in the vicinity of the greater tuberosity 30. This state-of-the-art repair is subject to a 20-60% failure rate, primarily due to suture tear-out through poor quality tendon tissue.

FIG. 5 shows an improvement to the repair of FIG. 4 with the addition of a patch 60 augmenting the repair. The edges 62 of the substantially planar patch 60 are attached to the rotator cuff tendon 20 by sutures 64. The edges 62 tend to pucker 66 when distorted over the approximately spherical tendon surface. The isotropic nature of the patch 60 results either in bulky excess material or insufficient strength along the direction of loading 68. The patch 60 is positioned on top of the sutures 65. The patch 60 does not contain any reinforced structure for attachment to the bone anchors 58 bone and the load on the sutures 65 is not transmitted through the patch 60.

In spite of numerous recent advances in primary fixation repair, 20-60% of rotator cuff repairs fail, primarily due to suture tear-out in poor quality tendon tissue. A number of factors affect the quality of the tendon tissue to be repaired: Patient age, health, physical condition and lifestyle choices, as well as the time delay between when the injury occurred and surgery. These factors present the surgeon with tissue ranging from thick, strong healthy tissue that is easily moved into apposition with the footprint, to thin, friable, connective tissue attached to retracted or atrophied muscle. The case of retracted tissue presents a particular challenge to the surgeon since tendon of poor quality must be placed in tension to move it into apposition with the footprint, making it particularly prone to failure.

A number of attempts have been made to provide materials and structures to strengthen or replace poor quality tendon and ligament tissue. These include non-absorbable polymer structures such as woven or knit mesh stitched over the tendon for reinforcement. This approach can provide structural reinforcement through out the healing period, but leaves behind a permanent device with all the abrasion, adhesion, migration and rejection issues associated with foreign bodies. Additionally, it has been shown that reinforcements that completely relieve the anatomic loads on the tendon or ligament lead to atrophy of the tissue. The tissue must experience some load in order to heal and strengthen.

To address these issues, researchers have reinforced ligaments and tendons with woven or knit structures made of commonly available absorbable polymers such as poly(glycolic acid) (PGA), poly(L-lactic acid) (PLLA) or PGA-PLLA copolymer blends. These structures are absorbed by the body and therefore eliminate permanent foreign body issues. They also gradually return anatomic loads to the ligament or tendon, thereby exercising and strengthening the tissue. However, the absorption characteristics of these materials can result in crystallization of the degrading polymer and acidification of surrounding tissue causing inflammation and tissue reactions. Further, most commonly available absorbable polymers loose most of their strength in 6 weeks or less, long before healing of the ligament or tendon-to-bone is complete.

In an attempt to overcome these shortcomings, a class of biologically derived implant materials have been developed. These materials include allografts, (e.g. Wright Medical GraftJacket™ [Human Dermis]) and xenografts, (e.g. Depuy Restore™ (Porcine SIS), Arthrotek Cuff Patch™ [Porcine SIS], Stryker TissueMend™ [Fetal Bovine Dermis], Zimmer Permacol™ [Porcine Dermis], Pegasus Orthadapt™ [Equine Pericardium], Kensey Nash BioBlanket™ [Collagen], CryoLife ProPatch™ [Bovine Pericardium]). In addition to providing structural reinforcement, these materials are intended to repopulate the host ligament or tendon tissue with appropriate ligament or tendon cells as they are absorbed by the body. However, recent research has revealed several shortcomings with biologically derived implants. First, though every attempt has been made to sterilize the material, infection and disease transmission has been observed. Second, even in sterile implants, foreign body reactions such as severe inflammation occur on a regular basis. Third, the tensile strength and elastic properties of most of these materials has been shown to be insufficient to provide any meaningful reinforcement. Fourth, most biomaterials have been shown to absorb long before healing is complete. Finally, while cell repopulation has been shown to occur, they tend to be mostly scar tissue and not the desired strong, highly oriented cellular structure of the host ligament or tendon tissue.

A common feature of all augmentation grafts to date is the use of substantially isotropic materials. Since the anatomic loads in ligament and tendons occur in distinct directions, corresponding to the anisotropic orientation of the cellular structure of the ligament or tendon itself, construction elements (filaments, cells, etc.) that are directional in nature and are not aligned with the tissue loads do not efficiently contribute to the strength of the device and only serve to bulk up the amount of foreign body material in the implant.

U.S. Pat. No. 5,441,508 (Gazielly et al.) discloses a reinforced rotator cuff patch having at least two divergent legs for fixation to at least two tendons. The ends of the patch are made semi-rigid mass by melting the component threads.

An additional aspect of the isotropic nature of the prior art is the need to withstand tear-out loads of suture stitches used to hold the graft in place. When used as a structural augmentation repair, the loads transmitted from the ligament or tendon to the implant through the sutures is substantial. It is estimated that in normal activities, the force transmitted through the cuff tendon is in the range from about 140 to about 200 Newtons (about 31.5 lbs to about 45 lbs). The ultimate tensile load of the supraspinatus tendon in specimens from the sixth or seventh decade of life has been measured between about 600 to about 800 Newtons (about 135 lbs. to about 180 lbs). Where the implant is homogeneous and isotropic, every portion of the implant must have sufficient material bulk to resist suture tear-out, even regions where sutures are not present. Again this results in unnecessary foreign body material bulk in the implant.

In addition to the inherent biologic burden associated with excess implant material, additional bulk impedes the ability of the implant to be manipulated in confined spaces, such as passing through an arthroscopic cannula. Many of the thicker implants are therefore limited to implantation by open surgery, rather than by less invasive arthroscopic surgical techniques.

Another common feature of the prior art is the substantially planar construction of the materials used. More often than not, the anatomical feature to be repaired is non-planar, and the implant is expected to conform to the feature. Again using the shoulder as an example, the tendonous structure of the rotator cuff is roughly spherical in shape. The result of stitching a planar augmentation graft to a roughly spherical tendon surface is localized puckering of the graft material, potentially resulting in impingement and interference with surrounding tissue.

### BRIEF SUMMARY OF THE INVENTION

The present invention relates to a method and implant for surgical repair of torn tendons and ligaments in the body, such as arthroscopic repair of torn rotator cuff tissue in the human shoulder.

The present method and implant relieves at least part of the separation forces experienced by the repair during the recovery period. The implant is preferably absorbed by the body after healing. The implant preferably distributes the separation forces experienced by a ligament or tendon-to-bone surgical repair during the recovery period over a large area of the ligament or tendon. The implant preferably includes reinforced regions in it construction to distribute attachment loads of sutures and prevent sutures from tearing through the device.

In one embodiment, the implant mimics the elastic properties of natural tendon in order to allow a portion of anatomical loads to stress the tendon and prevent atrophy of the attached muscle. In one embodiment, the implant conforms to the non-planar contours of an anatomical structure being repaired. The implant is preferably constructed in a shape that most effectively applies reinforcement loads in the anatomically correct orientation for the body part being repaired.

In another embodiment, the implant minimizes the foreign body material burden on the body by using an anisotropic construction with a majority of material filaments oriented in the direction of load bearing. By minimizing the foreign body material burden, the present implant elicits minimal foreign body tissue reactions such as inflammation or infection.

The present implant can be implanted using an open procedure or using minimally invasive arthroscopic surgical techniques.

In a preferred embodiment the present invention, the implant comprises a series of high strength, bioabsorbable filaments arranged to form a construct to conform in size, shape and orientation to, and align with, a tendon or ligament to be repaired. Different embodiments of this basic construct may conform to any size or shape ligament or tendon in the body, but for the purposes of illustration we shall use the supraspinatus tendon of the rotator cuff of the human shoulder as illustration.

A preferred embodiment of the present implant is roughly an isosceles trapezoid or flat-based fan shape in plan view with lateral, medial, anterior and posterior edges (relative to the device's intended orientation in the body. In a rotator cuff application, the lateral edge is reinforced to receive sutures and resist suture tear-out. The lateral edge is designed for fixation directly or indirectly to bone. The medial, anterior and posterior edges are also reinforced for stitch retention, but to a lesser extent than the lateral edge. The central portion of the construct consists of a series of filaments aligned with the direction of use experienced when used as a tendon augmentation, generally from the medial to lateral edges. The device is a relatively thin membrane formed to conform to the surface contour of the tendon to be repaired.

In the case of the rotator cuff this shape is roughly spherical. Some embodiments include a thin membrane or weave to hold the load carrying filaments in proper orientation, others have just the load carrying filaments between edges. Still other embodiments include a 3 dimensional matrix, such as a felt made of the implant material, to encourage cellular in-growth and repopulation with host tendon or ligament cells. Still other embodiments have filaments, membrane or 3D matrix infused with in-growth stimulants such as collagen-based matrices, glycosaminoglycans, heparin, chondroitin sulfate, hyaluronic acid, TCP, dermatan sulfate, chitin, chitosan, growth factors (including, but not limited to: PDGF, TGF-β, b-FGF, platelet lysates), fibrinogen/fibrin, thrombin, and oxidized cellulose/carboxymethyl cellulose.

Other embodiments have physical configurations achieving the same functional objectives. Some embodiments are substantially planar in their natural state and achieve contoured shape through elastic properties. Other embodiments include discrete load carrying strips or “fingers” that transmit distributed tendon loads to the lateral edge. Still other embodiments include self retaining features such at “T” toggles, barbs, hooks and the like. Still other embodiments have provision for suturing directly to the lateral edge through elongated suture strands, obviating the need for a mid-body structure. Still other embodiments include provision for combined or individual tension control on the load bearing filaments or sutures.

In use, the lateral edge is secured to bone, either by sutures, tack-like devices, staples, or any other devices known to the art for securing materials to bone. In some embodiments the lateral edge is secured to bone lateral to the lateral edge of the tendon. In other embodiments it is secured to bone through the tendon. The medial edge, and in some embodiments the anterior and posterior edges, and in still other embodiments the interior area enclosed by the edges too, are reinforced to receive stitches connecting the device to the tendon to be repaired. In so doing, the tendon is connected to the bone through the device through multiple stitch joints distributed over a large area of the tendon.

Tendon and ligament tissue is known to have unique biomechanical tensile and elastic properties. Properties vary between anatomical sites and individuals, but generally speaking tensile strength ranges from 50 to 150 MPa, modulus of elasticity ranges from 1.2 to 1.8 GPa, and tendons and ligaments experience a small hysteresis of 4-10% energy loss.

It is known that completely relieving the load from a tendon will eventually cause the tissue to atrophy. It is also known that, for the rotator cuff, allowing the tendon to experience full anatomical load during recovery will result in a 20-60% failure rate. The present method and implant provides a healing modality that shields the tendon from most of the anatomical loads in the early part of the recovery period, and gradually experience increasing loads as the repair heals to full strength. In an idealized repair, the combined strength of the augmentation implant and the healing surgical repair will equal the strength of the repaired tendon after full recovery.

The present invention achieves this goal through the use of materials and device design engineered to approximate the mechanical properties of natural tendon or ligament when first implanted, and then to degrade at approximately the same rate as the repair gains strength. Materials such as Poly-4-hydroxybutyrate (a.k.a.Tephaflex™), poly(urethane urea) (Artelon™), and surgical silk, to name a few, can be engineered to have tendon-like mechanical properties, are biocompatible, absorb over long periods of time, and elicit minimal detrimental tissue response during absorption.

One embodiment is directed to an implant for the repair of a tendon or a ligament along at least one load direction. The implant includes at least one first anchor portion and at least one tension member adapted to be oriented along a load direction. The tension member is secured to the first anchor portion with an overlapping attachment.

The first anchor portion preferably includes a first surface area of engagement greater than a second surface area of engagement of the tension member. In one embodiment, a second anchor portion is attached to the tension member offset from the first anchor portion. The tension member preferably comprises the sole portion of the implant located in a center region located between the first anchor portion and the second anchor portion.

At least one of the first anchor portion and the tension member preferably comprise a scalable weave. In one embodiment, the first anchor portion and/or the tension member comprise a bioabsorbable material with a strength retention after implantation of about 50% after about 2 months to about 50% after about 6 months.

In another embodiment the implant includes at least one first anchor portion and at least one second anchor portion offset from the first anchor portion. At least one tension member oriented along a load direction connects the first and second anchor portions. The tension member is connected to at least one of the first or second anchor portions with an overlapping attachment. A center region in the offset between the first and second anchor portions preferably includes more than 50% of the material located in the center region.

In another embodiment the implant includes a plurality of tension members comprising a radially distributed load profile corresponding generally to a plurality of load directions.

In another embodiment the tension member includes an elongated member laced through eyelets in the first anchor portion and the second anchor portion in a continuous loop. The tension members preferably comprises an equalized structure.

In another embodiment the implant includes a first discrete tension member oriented along a first load direction with a first end adapted to engage with the first bone anchor. A second tension member is optionally included with a first end adapted to engage with the first bone anchor along a second load direction.

In another embodiment the implant includes a patch material with a first edge and a second edge. At least one elongated slot is located in the patch material between the first and second edges. A suture material is laced along opposite edges of the elongated slot so that tension on the suture material reduces the elongated slot and increases tension between the first and second edges along a load direction.

In another embodiment the implant includes a first layer comprising a plurality of protrusions adapted to penetrate a tendon or a ligament and a second layer adapted to engage with distal ends of the protrusions on the other side of the tendon or ligament. At least one first anchor portion is offset from the first and second layers and a tension member connects the first and second layers to a first anchor portion.

In another embodiment the implant includes at least one tension adjusting device adapted to adjust tension on the tension member.

In another embodiment the implant comprises a patch material of a scalable weave or a material with at least one pre-determined cut line.

The present invention is also directed to a method of repairing a tendon or a ligament including the steps of attaching at least one first anchor portion to tendon, ligament or bone. At least one tension member secured to the first anchor portion with an overlapping attachment is oriented along a load direction. Distal ends of the tension member are attached to tendon, ligament or bone.

### BRIEF DESCRIPTION OF THE SEVERAL VIEWS OF THE DRAWING

FIG. 1 is a posterior-lateral anatomical view of an anatomical human shoulder.

FIG. 2 is a posterior-lateral anatomical view of a left human shoulder with a torn supraspinatus tendon.

FIG. 3 is a posterior anatomical view of a right human shoulder with a torn supraspinatus tendon.

FIG. 4 is a posterior-lateral anatomical view of a left human shoulder with a prior art arthroscopic repair of a torn suprasinatus tendon.

FIG. 5 is a posterior-lateral anatomical view of a left human shoulder with a prior art patch repair of a torn suprasinatus tendon.

FIG. 6 is a posterior-lateral anatomical view of a left human shoulder with an implant for repairing a torn tendon in accordance with an embodiment of the present invention.

FIG. 7 is a graphical illustration of an idealized tendon repair.

FIGS. 8a-8c are additional view of the implant illustrated in FIG. 6.

FIGS. 9a-9b are views of an alternate implant for repairing a torn tendon in accordance with an embodiment of the present invention.

FIG. 10 illustrates an implant for repairing a torn tendon with a plurality of discrete tension members in accordance with an embodiment of the present invention.

FIG. 11 illustrates an implant for repairing a torn tendon with a plurality of filament-based tension members in accordance with an embodiment of the present invention.

FIG. 12 illustrates an implant for repairing a torn tendon with a plurality of suture-based tension members in accordance with an embodiment of the present invention.

FIG. 13 illustrates an implant for repairing a torn tendon with off-set reinforced pads in accordance with an embodiment of the present invention.

FIGS. 14, 15 and 16 illustrate an implant for repairing a torn tendon with a single load direction in accordance with an embodiment of the present invention.

FIG. 17 illustrates an implant with a load-spreading patch for repairing a torn tendon in accordance with an embodiment of the present invention.

FIG. 18 illustrates an implant for repairing a torn tendon with integral tensioning mechanisms in accordance with an embodiment of the present invention.

FIGS. 19a-19c illustrate an implant for repairing a torn tendon with discrete tension members in accordance with an embodiment of the present invention.

FIGS. 20a-20c illustrate a self-equalizing tension member for repairing a torn tendon in accordance with an embodiment of the present invention.

FIG. 21 illustrates an implant for repairing a torn tendon with self-equalizing tension members in accordance with an embodiment of the present invention.

FIG. 22 illustrates an implant for repairing a torn tendon with multiple lateral anchor locations in accordance with an embodiment of the present invention.

FIG. 23 is a side view of the lateral anchor locations for the implant of FIG. 22.

FIG. 24 is a side view of the lateral portion of the implant of FIG. 22 engaged with a bone anchor.

FIG. 25 is a posterior anatomical view of a right human shoulder illustrating an implant for repairing a torn tendon with infinitely adjustable anchor locations in accordance with an embodiment of the present invention.

FIGS. 25b-25c are an anatomical view of a right human shoulder illustrating an implant for repairing a torn tendon with infinitely adjustable anchor locations in accordance with an embodiment of the present invention.

FIGS. 26 and 27 illustrate an alternate implant threaded through one or more slits in a tendon in accordance with an embodiment of the present invention.

FIG. 28 illustrates an alternate implant with loops at the first and second end in accordance with an embodiment of the present invention.

FIG. 29 illustrates an alternate implant comprising a loops in accordance with an embodiment of the present invention.

FIGS. 30a and 30b are perspective views of a rotator cuff repair using an implant in accordance with an embodiment of the present invention.

FIGS. 31a-31b illustrate an implant with an attachment mechanism to tissue in accordance with an embodiment of the present invention.

FIG. 32 is a side view of an alternate tissue attachment mechanism in accordance with an embodiment of the present invention.

### DETAILED DESCRIPTION OF THE INVENTION

The present method and apparatus can be used to repair and reconstruct torn ligaments and tendons in a variety of locations of the body. The rotator cuff muscles were selected for the exemplary embodiments because of the complexity of the human shoulder. It will be appreciated that the following method and apparatus has many other possible applications.

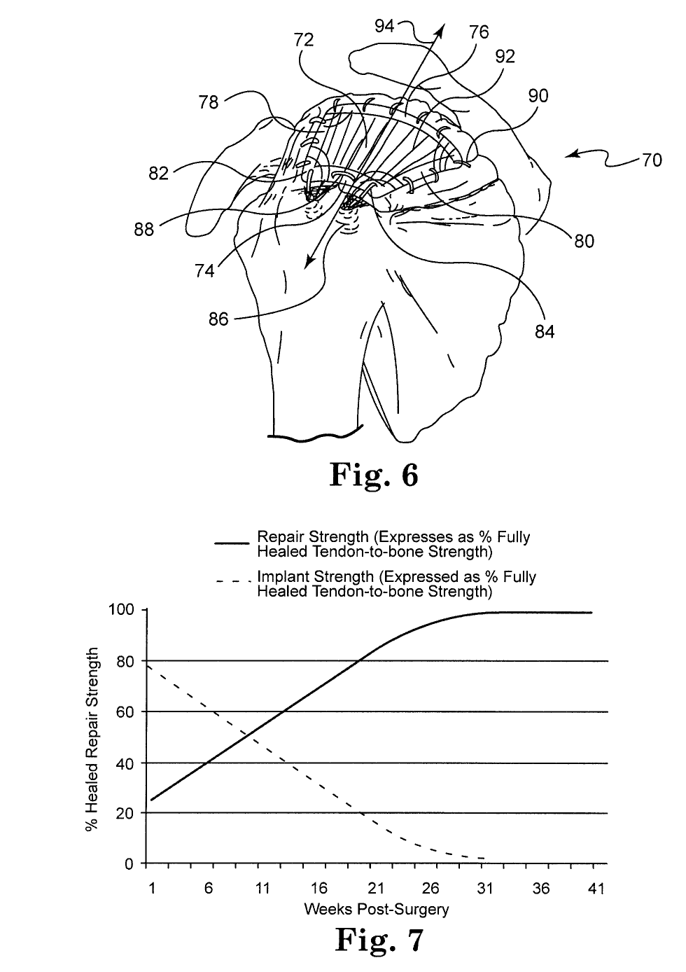

FIG. 6 shows a bioabsorbable implant 70 made from a patch material 72 in accordance with an embodiment of the present invention. Implant 70 includes first and second edges 74, 76, and first and second side edges 78, 80. In a rotator cuff application, the implant 70 includes lateral edge 74, medial edge 76, anterior edge 78, and posterior 80 edges. The lateral edge (i.e., first edge) 74 and medial edge (i.e., second edge) 76 are preferably reinforced to facilitate attachment. The anterior 78 and posterior edges 80 (i.e., the side edges) are also optionally reinforced.

In some embodiments the anterior and posterior edges 78, 80 are symmetrical and may be reversed for use in the right shoulder. In other embodiments the implant 70 is asymmetrical and would be manufactured in specific left and right versions. The terms lateral and medial used as modifiers for “edge”, “end” or “portion” refer to the first and second edge, end or portion of the implant, respectively, in the rotator cuff context. For purposes of this application, however, the terms lateral and medial should be broadly construed to mean the first and second edge, end or portion of the implant, without regard to a particular location or orientation within the body.

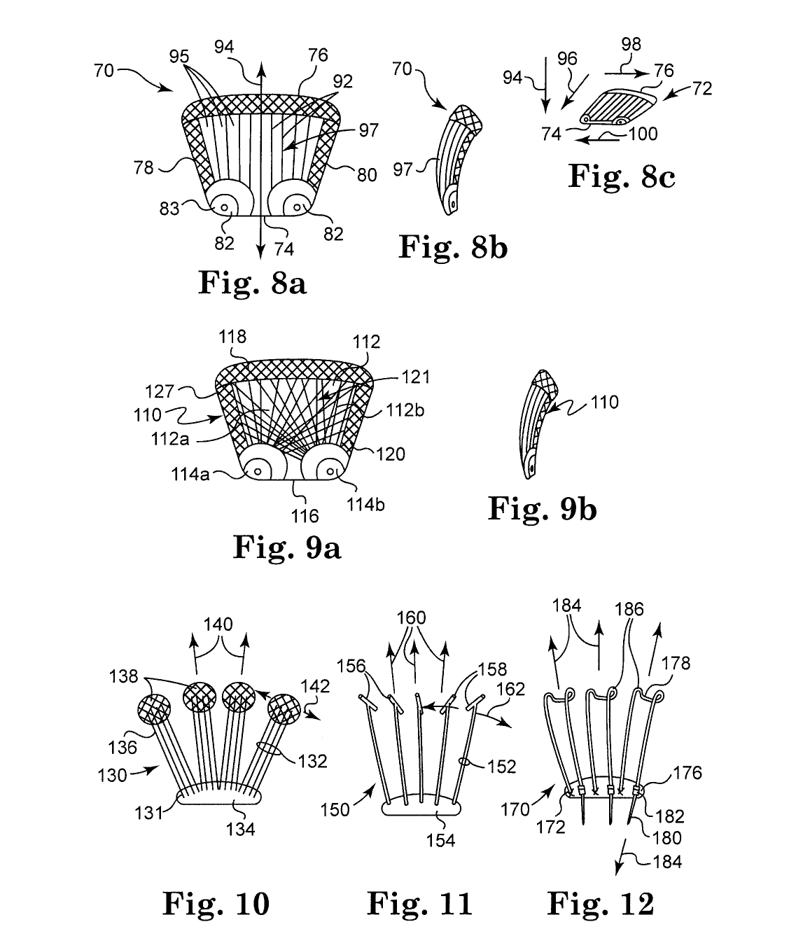

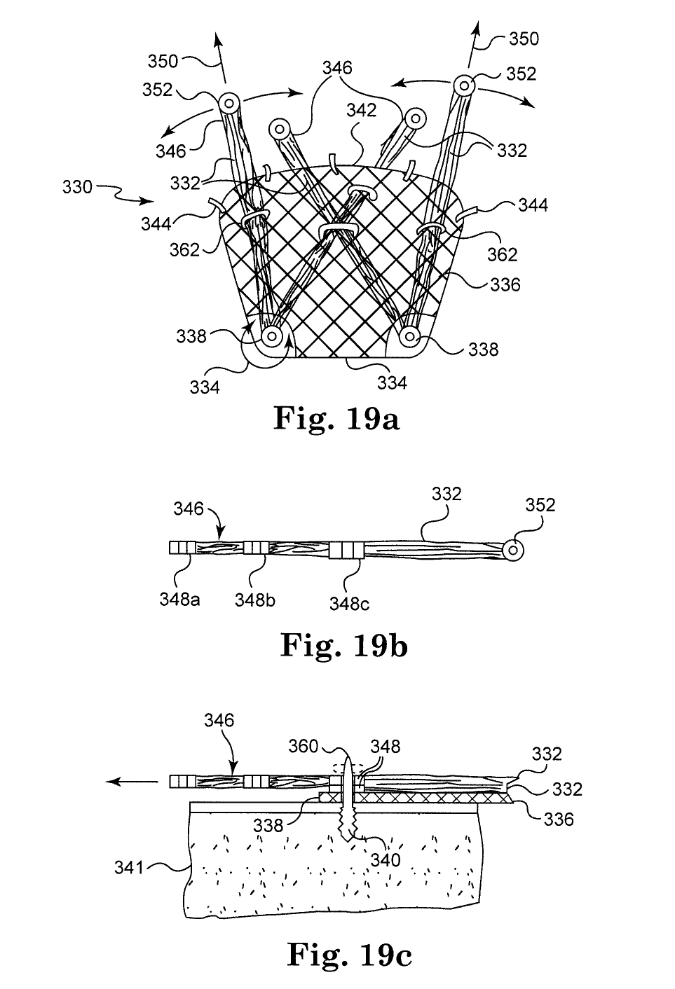

The lateral edge 74 preferably includes anchor portions 82 that resist tear-out of sutures 84 attached to bone through bone anchors 86 and/or through other attachment mechanisms, such as trans-tendon anchors 88, darts, screws, glue, tacks, staples, or any combination thereof. Various attachment mechanisms suitable for the present method and apparatus are disclosed in U.S. Pat. Nos. 6,923,824; 6,666,877; 6,610,080; 6,056,751; and 5,941,901, which are hereby incorporated by reference. As best illustrated in FIG. 8a, the anchor portions 82 optionally include a pre-formed opening or eyelet 83 adapted to receive sutures 84 or to engage with another anchoring structure, such as for example a bone anchor (see e.g., FIG. 19c). Eyelet refers to a preformed opening, preferably round and finished along the edge.

In the illustrated embodiment, the lateral, medial, anterior and posterior edges 74, 76, 78, 80 are reinforced to resist suture tear-out and to increase strength. In one embodiment, reinforcement includes welding of the patch material 72 along one or more of the edges. The welding can be performed with various types of energy, such as, for example, ultrasonic, laser, electrical arc discharge, and thermal energy. In another embodiment, multiple layers of patch material 72 are attached to one or more of the edges, such as by adhesive, welding, or mechanical fasteners. In another embodiment, additional layers of patch material 72 and/or tension members are woven or knitted along the edges. Sutures 90 in the medial, anterior and posterior edges 76, 78, 80 distribute tendon loads to the bone, through the lateral edge 74, by use of high strength tension members 92 arranged along the preferred load direction 94.

The patch material and tension members may be the same or different material/structure. For example, the patch material may be bioabsorbable, while the tension members include a non-absorbable component. In another embodiment, the patch material and/or the tension members are a composite of synthetic material and natural material, such as for example an allograft or a xenograft materials.

In yet another embodiment, the patch material and/or tension members are multiple component materials. The different materials may each have a different melting point. The patch material and/or tension members can be composed of a single filament or multiple filaments. The filaments can be homogenous or heterogeneous. When multiple filaments are present, the material composition of the filaments can vary from filament to filament. Multiple filaments can include a mixture of both single-material filaments and multi-material filaments. The patch material and/or tension members may be a single strand of multiple fibers or it can include multiple strands. When multiple strands are included, these may be twisted together, braided or otherwise interlinked, such as in a sheath-and-core configuration. A composite material for use in the present method and apparatus is disclosed in U.S. Patent Publication No. 2007/0021780, filed Jun. 5, 2006, which is hereby incorporated by reference.

The structure of the patch material and tension members can be one or more layers of the same or different materials, such as for example woven mesh, non-woven mesh (such as for example melt-blown, hydro-entangled, etc.), multifilament mesh, monofilament mesh, terrycloth, fabric made by weaving, knitting, braiding or felting fibers, film, or any combination or composites thereof. Patch material and tension members may also be autologous, allogeneic, or xenogeneic tissues.

In some embodiments, the tension members are a single filament such as a monofilament, or a grouping of a plurality of pliable, cohesive threadlike filaments (e.g., braided suture), an elongated section of woven or non-woven fabric or mesh.

The patch material and the tension members disclosed herein are preferably made of an absorbable or bioabsorbable material. As used herein, “absorbable” or “bioabsorbable” means the complete degradation of a material in vivo, and elimination of its metabolites from an animal or human. Bioabsorbable implants according to an embodiment of the present invention preferably have a strength retention after implantation of about 50% after 2 month to about 50% after about 6 months, and preferably about 50% after 4 months.

While the patch material and tension members are preferably constructed from an absorbable material, one or both may be reinforced by non-absorbable materials, including without limitation glass fibers, natural fibers, carbon fibers, metal fibers, ceramic fibers, synthetic or polymeric fibers, composite fibers (such as a polymeric matrix with a reinforcement of glass, natural materials, metal, ceramic, carbon, and/or synthetics components), or a combination thereof. In one embodiment the tension members are relatively stiff and the implant does not distort in response to oblique loads. In other embodiments the tension members are somewhat elastic and the implant distorts under oblique loads.

In one embodiment, the tension members and patch material comprise a woven structure that can be trimmed to shape with minimal or no unraveling/fraying along the cut edge. Fraying reduces mechanical strength and suture retention ability. Loose fibers can migrate and provoke an inflammatory reaction. Consequently, special textile manufacturing techniques may be used to prevent these problems.

In one embodiment, the patch material and/or tension members are constructed using a ‘leno’ weave. A standard weave has an array of warp fibers in one direction and the weft fibers run alternately over and under them in the perpendicular direction. In a leno weave the weft fibers wrap over and under each warp fiber and lock each fiber in place. Leno weaves are much more resistant to unraveling when cut. Leno weave are also more porous and allow tissue in-growth better than plain weaves.

In another embodiment, the tension members and/or patch material comprise a weave constructed on a shuttle loom. In modern, conventional weaves the weft fibers run across the fabric and end at the edges. With a shuttle loom the weft fiber is woven across the fabric and then it turns around and weaves back across the fabric as a continuous fiber. The edges are thus much more stable. As used herein, “scalable weave” refers to a textile structure that can be trimmed with minimal or no unraveling along cut edges, such as for example by a leno weave or textiles made using a shuttle loom.

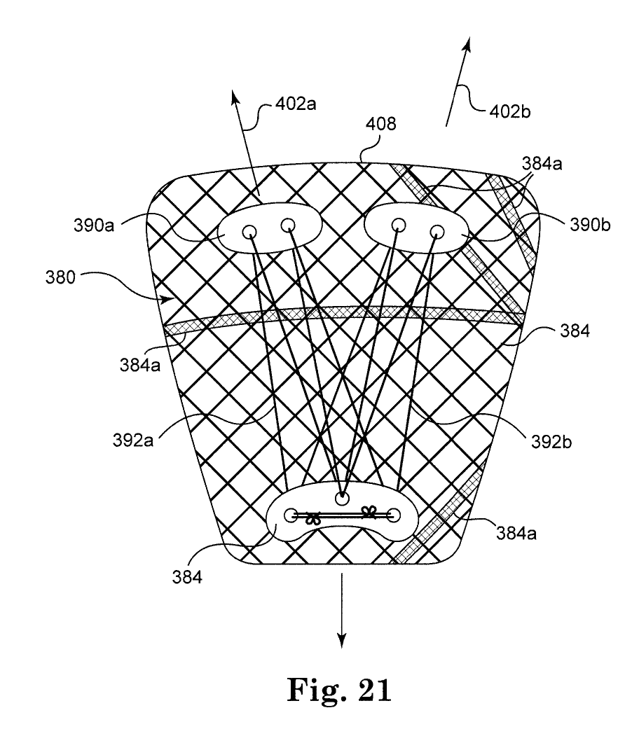

While the scalable weaves discussed above are infinitely scalable (i.e., the surgeon can cut anywhere with minimal risk of fraying or unraveling), in some embodiments the patch material and/or tension members are reinforced to be incremental scalable (i.e., the surgeon can cut along pre-determined cut lines). Pre-determined cut lines be formed by welding, adding a resin to the fabric, attaching one or more reinforcing layers, or combinations thereof (see e.g., FIG. 21). The surgeon can cut the patch material and/or tension members along the pre-determined cut lines to fit a particular patient with minimal risk of fraying or unraveling.

In a preferred embodiment the patch material and the tension members are made of a slow-absorbing, biologically benign material, such as Poly-4-hydroxybutyrate (a.k.a.Tephaflex™), poly(urethane urea) (Artelon™), surgical silk, polymers containing lactide, glycolide, caprolactone, trimethylene carbonate or dioxanone, or other materials, known to the art, having similar characteristics, such as disclosed in U.S. Patent Publication No. 2007/0198087, entitled Method and Device for Rotator Cuff Repair, filed Feb. 5, 2007 and U.S. Patent Publication No. 2007/0276509, entitled Tissue Scaffold, filed Aug. 9, 2007, the entire disclosures of which are incorporated by reference. Other less preferred embodiments employ non-absorbable materials such as PTFE, Polyester, Polypropylene, Nylon, or other biocompatible, inert materials known to the art. In some embodiments, monofilaments may be used in combination with weaves, knits, braids, etc, to increase porosity for tissue in-growth and selective tensile strength along a load direction. Alternatively, the implant may be constructed from xenograft and/or allograft materials.

Immediately after surgery, the implant 70 carries the majority of the anatomical loads through the distributed interface of the medial, anterior and posterior edges 76, 78, 80, though the tension members 92 to the lateral edge 74. In another embodiment, the implant 70 load shares through the distributed interface of the medial, anterior and posterior edges 76, 78, 80, though the tension members 92 to the lateral edge 74. During the course of healing (usually 26 to 52 weeks) the tendon-to-bone repair gains strength while the augmentation implant 70 loses strength and is absorbed by the body.

While the surgical repair has historically been performed as an open procedure (and more recently as a “mini-open” repair), the majority of rotator cuff repairs are now repaired fully arthroscopically, with the tendon being reattached directly to the bony insertion on the lateral border of the humerus. However, when direct reattachment is not possible, for example, because retraction of the muscle has created a large defect, interposition implants or grafts (including synthetic cuff prostheses) are used to fill the gap formed by the defect. Implants (or grafts) are also used as augmentation implants to strengthen a repair to prevent recurrent tears and allow for a more aggressive rehabilitation particularly in younger patients. Augmentation implant (or graft) refers to a material that can be used to strengthen a tendon or ligament. For example, a surgeon may enhance the strength of a rotator cuff repair made with sutures by incorporating a reinforcing material into the repair. Interposition implant (or graft) refers to a material that is used to bridge a gap (or defect) between the end of a tendon and its bony insertion site. As used herein, “implant” refers to at least an interposition implant and/or an augmentation implant.

FIG. 7 shows a graph of an idealized augmented rotator cuff repair scenario. The strength to the surgical repair, expressed as percent strength of the final healed repair, begins post-surgically at the strength of the suture-to-tissue connection alone. In this illustrated example, the suture-to-tissue connection represents about 25% of the strength. The augmentation implant initially receives the majority of the loads experienced during recovery through high initial strength, in this embodiment about 75%. Gradually, the ratio of load sharing shifts to the suture-to-tissue connection as the repair heals and gains strength, while the implant is simultaneously absorbed by the body. Strength retention refers to the amount of strength that a material maintains over a period of time following implantation into a human or animal. For example, if the tensile strength of an absorbable mesh or fiber decreases by half over three months when implanted into an animal or human, the mesh or fiber's strength retention at 3 months would be 50%.

FIGS. 8a through 8c illustrate various views of the implant 70 illustrated in FIG. 6. In the illustrated embodiment, the tension members 92 are oriented generally parallel. The primary load direction of the implant 70 is along the load direction 94. The illustrated configuration reduces the overall volume (i.e., bulk) and thickness of the implant 70 by concentrating the material along a primary load direction 94.

Tension members 92 are preferably positioned in substantially directly side-by-side, non-overlapping, slightly spaced relationship to form a blanket of fibers without substantial breaks there between. As used herein, “non-overlapping” refers to generally coplanar fibers that do not extend over or cover one another.

In one embodiment, the tension members 92 are incorporated into the patch material 72 in center region 97 extending between two or more of the reinforced edges 74, 76, 78, 80. The patch material 72 provides longitudinal strength, shear strength and anti-skew properties to maintain the relative orientations and/or spacing of the tension members 92 when the implant 70 is subjected to oblique load directions arising during twisting action of the anatomy. The tension members 92 preferably comprise more than 50%, and more preferably at least 75%, of the total volume of material comprising the center region 97 of the implant 70. In another embodiment, the patch material 72 is reinforced in the center region 97, using one or more of the techniques discussed above.

In another embodiment, the tension members 92 are the sole material of the implant 70 extending through the center region 97 (i.e., between the medial edge 76 and the lateral edge 74). In an embodiment where the tension members 92 are the sole material of the implant extending between the medial edge 76 and the lateral edge 74, the tension members 92 in the center region 97 comprise a thickness of less than about 5 millimeters and preferably less than about 2 millimeters, and most preferably less than 0.5 millimeters. The tension members 92 preferably comprise at least 30%, and more preferably at least 70%, of the total volume of material comprising the implant 70.

As illustrated in FIG. 8b, the implant 70 is preferably formed with a roughly spherical contour corresponding generally to the curvature of the shoulder anatomy. A plurality of implants 72 with different curvatures are preferably provided to the surgeon so the optimum size can be selected.

FIG. 8c is a perspective view shows a plan view of the same embodiment where the high strength filaments 92 subject to an oblique load direction 96 that differs from the load direction 94. Oblique load directions are characteristic twisting action of the anatomy, such as twisting of the arm. In this embodiment the implant 70 responds by distortion in a racking direction, where the lateral edge 74 and medial edge 76 remain substantially parallel while moving in opposite directions 98 and 100.

FIGS. 9a and 9b illustrate an alternate implant 110 in accordance with another embodiment of the present invention. The high strength tension members 112 are arranged in a radial configuration. In particular, a plurality of tension members 112a extend radially from anchor portion 114a at the lateral edge 116 toward the medial edge 118 and posterior edge 120 at a plurality of angles. The radial distribution of the tension members 112a is preferably between about 120 degrees to about 20 degrees. The tension members 112a preferably fan-out uniformly from the anchor portion 114a, although a non-uniform distribution of tension members 112a is possible for some applications.

A plurality of tension members 112b also extend radially from anchor portion 114b at the lateral edge 116 toward the medial edge 118 and the anterior edge 122 at a plurality of angles. The tension members 112a, 112b are preferably overlapping to minimize thickness of the implant 110, although the tension members 112 may be interwoven for some applications. The structure of the implant 110 distributes the lateral edge loads more uniformly over the medial, posterior and anterior edges 118, 120, 122 edges. The embodiment of FIGS. 9a and 9b mimics the splaying out and interdigitated nature of the rotator cuff 20. The tension members 112 preferably comprise more than 50%, and more preferably at least 75%, of the total volume of material comprising the center region 121 of the implant 110.

The tension members 112a and 112b each provide a radially distributed load profile. As used herein, a “radially distributed load profile” means a plurality of tension members extending generally radially from a common location. The implant 110 provides two discrete radially distributed load profiles extending from two different locations on the lateral edge 74 to at least the medial edge 76.

FIG. 10 is a plan view of an implant 130 in accordance with another embodiment of the present invention. First ends 131 of discrete tension members 132 are attached to first anchor portion 134. The first anchor portion 134 preferably has a greater surface area of engagement with the tendon or ligament than the tension members 132. The tension members 132 are preferably attached along a portion of the surface area of the first anchor portion 134. In particular, the first ends 131 preferably form an overlapping attachment with the first anchor portion 134 to distribute loads over a greater surface area.

As used herein, “overlapping attachment” refers to common surface areas between two members along which at least a portion of an attachment is located. An overlapping attachment involves greater surface area than is achieved when a narrow suture penetrates an un-reinforced patch material. Overlapping attachments may be used in combination with other attachment structures, including suture stitches. Actual attachment between the two members can be achieved using various techniques, including without limitation, adhesives, fasteners, mechanical interlocks, interwoven structures, welding, integrally formation as a unitary structure, co-extrusion, or combinations thereof.

The second ends 136 of each of the tension members 132 preferably include an anchor portion 138 designed to receive sutures and resist tear-out. The second ends 136 also preferably form an overlapping attachment with the anchor portions 138. In the illustrated embodiment, the tension members 132 are a plurality of filaments.

The discrete tension members 132 can be configured in any parallel or non-parallel configurations. The load direction 140 of each tension member 132 can be adjusted independently in direction 142 by the surgeon prior to attachment of the anchor portion 138. Some embodiments of the implant 130 are pre-formed to conform to the ligament or tendon being repaired. Other embodiments are formed flat and will approximately conform due to the flexibility of the individual tension members 132.

FIG. 11 is a plan view of an implant 150 in which tension members 152 are monofilaments or bundles of multifilament fibers attached to a reinforced first anchor portion 154. The first anchor portion 154 has a greater surface area of engagement with the tendon or ligament than the tension members 152. The tension members 152 are preferably attached to the first anchor portion 154 by an overlapping attachment. The second ends 156 are equipped with retaining feature 158 designed to be inserted through the tendon to form an attachment point. The retaining features 158 can optionally be barbs, hooks, buttons, and a variety of other devices known in the art.

The load direction 160 of each tension member 152 can be adjusted independently in directions 162 by the surgeon prior to attachment. The monofilament or fiber structure of the tension members 152 permit a greater degree of adjustment in directions 162 than the embodiment of FIG. 10. In some embodiments, the tension members 152 can be arranged in an overlapping configuration.

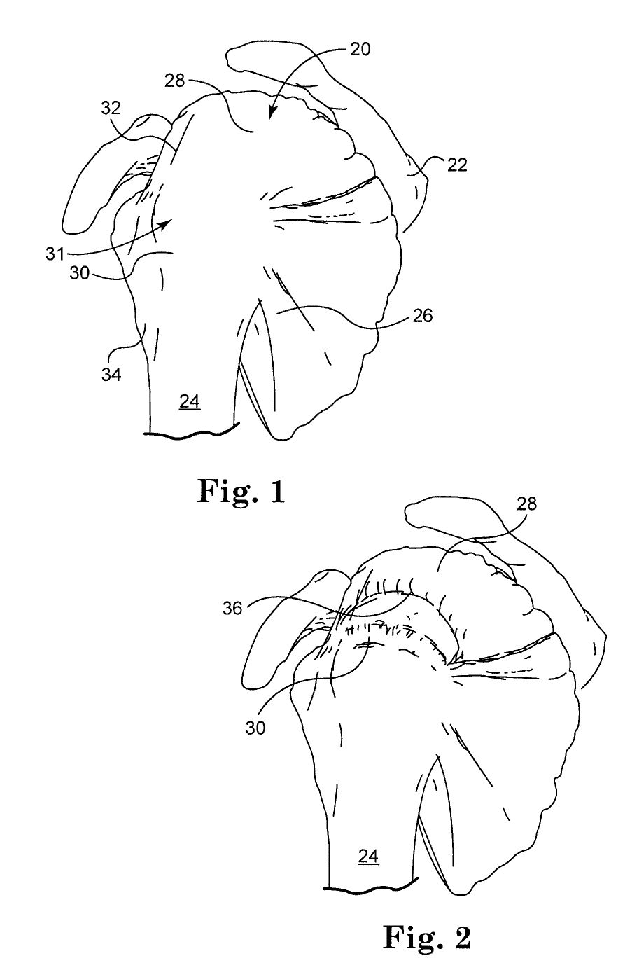

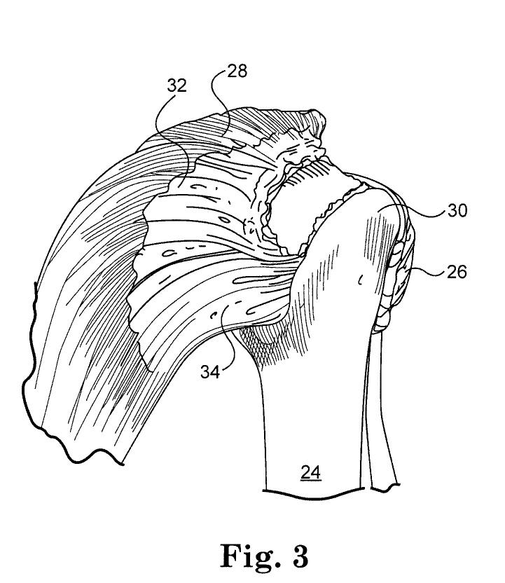

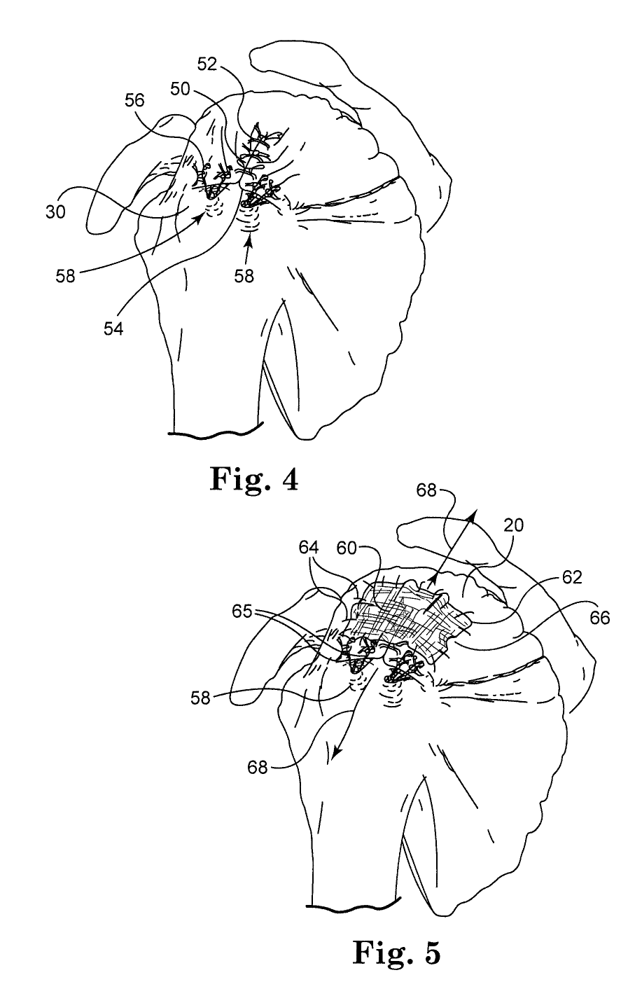

In one embodiment, the reinforced first anchor portion 154 is attached to a rotator cuff tissue using a plurality of sutures around the perimeter. The size and shape of the reinforced first anchor portion 154 permits the tension load to be distributed over a greater area than in prior rotator cuff patches. The retaining features 158 are then attached to the greater tuberosity 30 (see FIG. 1) or a lateral portion of the tendon.

FIG. 12 is a plan view of an implant 170 in accordance with an embodiment of the present invention. First end 172 of each of tension members 174 is attached to portion 176, preferably with an overlapping attachment. In use, once the anchor portion 176 is affixed to bone, the second end 180 of the tension members 174 is passed through a tendon to be repaired using a suitable stitch 178, such as for example a mattress stitch. The second end 180 is then inserted in situ through a tension holding device 182. The tension holding device 182 can be any of a variety of friction, ratcheting, or clutch mechanisms known to the art, such as for example a cable tie structure. The surgeon tensions the implant 170 by pulling on second end 180 in the direction 184. Consequently, the tension and load direction 184 of each tension members 174 can be adjusted independently. In an alternate embodiments both ends of the tension member 174 are engaged with the portion 176 using tension holding devices 182, allowing the surgeon to tension both sides of the stitch 178.

In an alternate embodiment, the anchor portion 176 is attached to tissue using a plurality of sutures around the perimeter. The size and shape of the anchor portion 176 permits the tension load to be distributed over a greater area. The second end 186 of the implant 170 is attached to tissue or bone. The surgeon then applies tension to the implant 170 as discussed above.

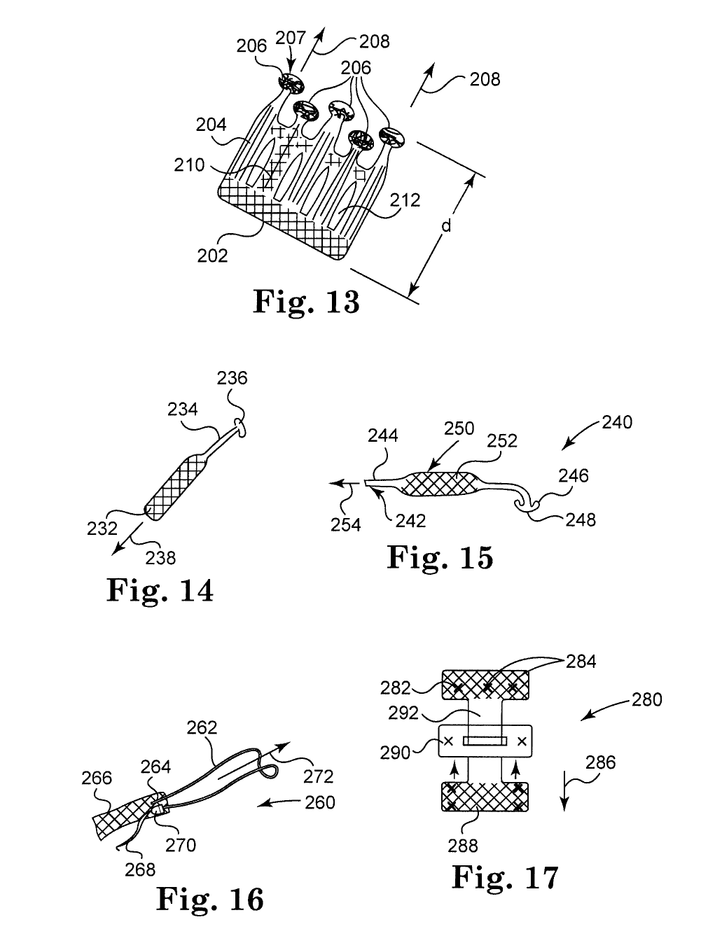

FIG. 13 is a plan view of an implant 200 in accordance with an embodiment of the present invention. First edge 202 includes a plurality of tension members 204 connected to reinforced pads 206 at second end 207. The reinforced pads 206 are disposed at variable distances “d” from the first edge 202 to distribute stress at different levels of the tendon or ligament.

In addition to the high strength tension members 204 oriented in particular load directions 208, a matrix or substrate of smaller fibers 210 provide a backplane to stabilize the tension members 204 and provide interstices for tissue in-growth and host tissue cell repopulation. In some embodiments the matrix of smaller fibers 210 are an organized weave or knit mesh if filaments of the implant 200 material. Other embodiments are comprised of a non-woven construction such as a felt. The implant optionally includes gaps 212 between the tension members 204 to reduce material volume of the implant 200 or further encourage tissue in-growth.

FIG. 14 is a plan view of an implant 230 in accordance with another embodiment of the present invention. Reinforced strip 232 is affixed to a high strength tension member 234 which terminates in a retaining feature 236 designed to be inserted through the tendon to form an attachment point. The reinforcing strip 232 is preferably integrally formed with the tension member 234 so both portions comprise a unitary structure.

In practice, the retaining feature 236 is affixed to the ligament or tendon to be repaired, and the reinforced strip 232 is tensioned in the opposite direction (for the rotator cuff) by the surgeon and affixed, under tension, to bone using sutures, bone anchors, staples, or other soft-tissue-to-bone fixation devices known to the art. The surgeon may use a single implant 230, or multiple implants 230 to distribute tendon loads to bone. The implant 230 can be oriented in any load direction 238 relative to the retaining feature 236 elected by the surgeon.

In an alternate embodiment, the reinforced strip 232 is affixed to the ligament or tendon. The reinforced strip 232 has a greater surface area of engagement with the tendon than the tension member 234, distributing loads over a greater area of tissue. The retaining features 236 is then affixed under tension to the bone using sutures, bone anchors, staples and the like. One advantage of this embodiment is that multiple sutures can be used to attach the reinforced strip 232 to the ligament or tendon, thereby distributing the load over a greater area of tissue.

FIG. 15 is a plan view of an implant 240 in accordance with another embodiment of the present invention. The first end 242 of the implant 240 terminates in a suture strand 244, which may be tied to another suture or other structure which is fastened to bone. The second end 246 terminates in a retaining feature 248. In some embodiments the middle portion 250 of the implant 240 includes an anchor portion 252, which may receive sutures, anchors or other fixation means to cause the implant 240 to hold the ligament or tendon to be repaired in apposition to bone. The implant 240 can be oriented in any load direction 254 relative to the retaining feature 248 elected by the surgeon.

FIG. 16 is a plan view of an implant 260 in accordance with another embodiment of the present invention. A segment 264 of high strength tension member 262 is attached to reinforcing strip 266. The segment 264 provides an overlapping attachment that distributes load across a larger surface area of the reinforcing strip 266. The overlapping attachment of segment 264 is preferable over a point attachment, such as for example a suture stitch penetrating the reinforcing strip 266.

In use, the reinforced strip 266 is affixed to bone and the second end 268 of the tension member 262 is passed through the ligament or tendon to be repaired, as discussed above. The surgeon adjusts tension on the tissue to be repaired by pulling on the second end 268 through tension holding device 270. The surgeon may use a single implant 260, or multiple implants to distribute tendon loads to bone. The implant 260 can be oriented in any load direction 272 elected by the surgeon.

In an alternate embodiment, a plurality of sutures extending around the reinforced strip 266 are used to affix the implant 260 to the ligament or tendon, thereby distributing the load over a greater area of tissue. The reinforced strip 266 has a greater surface area of engagement with the tendon than the tension member 262, distributing loads over a greater area of tissue. The tension member 262 is affixed to the bone using sutures, bone anchors, staples and the like. Once both ends 266, 262 are attached, the surgeon can apply tension by pulling the second end 268 of the tension member 262 through the holding device 270.

FIG. 17 is a plan view of an implant 280 in accordance with another embodiment of the present invention. Reinforced second end 282 is affixed to tissue by a plurality of attachment points 284. Once the reinforced second end 282 is attached, the surgeon applies tension in the direction 286 on a reinforced first end 288. Once the desired tension is achieved, the reinforced first end 288 is affixed to bone. A load spreading patch 290 is free to slide on a tension carrying strip 292. The load spreading patch 290 is positioned over the “footprint” and fastened to bone through the tendon to approximate the healing surfaces of bone and tendon for reattachment.

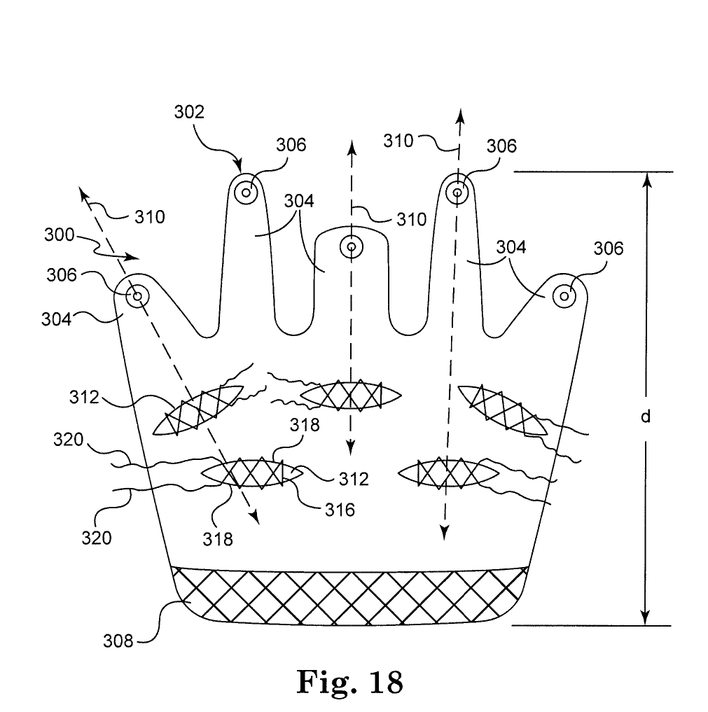

FIG. 18 is a plan view of an implant 300 in accordance with an embodiment of the present invention. Second edge 302 includes a plurality of tension members 304 connected to reinforced pads 306. The reinforced pads 306 are disposed at variable distances “d” from the first edge 308 to distribute stress at different levels of the tendon or ligament. In the illustrated embodiment, the tension members 304 are oriented in a variety of different load directions 310.

The implant 300 optionally includes a plurality of elongated gaps 312a-312e (collectively “312”) located between the tension members 304 and the first edge 308. The elongated gaps 312 are preferably oriented perpendicular one of the load directions 310.

Suture material 316 is laced to edges 318 of each elongated gap 312. By tensioning to the free ends 320 of the suture material 316 the surgeon can reduce or close the elongated gap 312, thereby applying tension along one of the load directions 310. Once the desired level of tension is achieved, the surgeon ties-off the free ends 320 of the suture material 316. The elongated gaps 312 also reduce material volume of the implant 300 and encourage tissue in-growth.

FIG. 19a-19c illustrate various views of an implant 330 with discrete tension members 332 in accordance with an embodiment of the present invention. First edge 334 of patch material 336 includes one or more eyelets 338 that engaged with bone anchors 340 secured to bone 341 (see FIG. 19c). The second edge 342 of the patch material 336 is preferably attached to the tendon with sutures 344.

First ends 346 of the tension members 332 include eyelets 348 that pivotally engage with a bone anchor 340. The tension members 332 are free to rotate along path 339 around the bond anchor 340 to a desired load direction 350. The second ends 352 of the tension members 332 are then attached to the tendon at the desired location.

As illustrated in FIGS. 19b and 19c, the tension members 332 optionally include a plurality of eyelets 348a-348c near the first end 346. Once the second end 352 of a tension member 332 is attached to the tendon, the surgeon has the option to engage any of the eyelets 348a-348c with the bone anchor 340 to increase or decrease tension on the tension member 332.

Once the desired eyelet 348a-348c is selected, the distal end 360 of the bone anchor 340 is optionally deformed (shown in dashed lines) to secure the tension member 332. As best illustrated in FIG. 19c, the shape of the distal end 360 of the bone anchor 340 to form an overlapping attachment with the eyelets 348. The bone anchor 340 can be deformed by thermal or ultrasonic energy, mechanical deformation, or a variety of other methods known in the art. The unused first ends 346 of the tension member 332 are then removed. The eyelets 348 can optionally serve as pre-determined cut lines to minimize fraying or unraveling of the tension member 332. The tension members 332 are optionally attached to the patch material 336 and/or the tendon with sutures 362.

The modular construction of the tension members 332 permits the surgeon to select from reinforcing structures 332 of different lengths and diameters, to rotate the reinforcing structure 332 relative to the bone anchor 340 to the desired load direction 350, and to locate multiple reinforcing structures on a single bone anchor 340. In an alternate embodiment, the reinforcing structures 332 are used without the patch material 336.

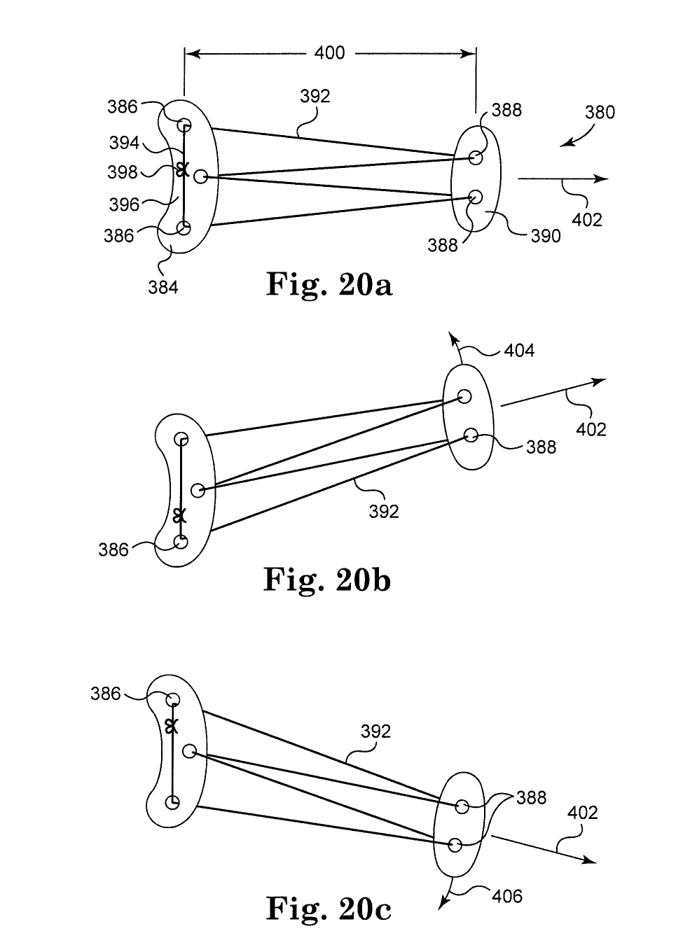

FIGS. 20a-20c illustrate an alternate implant 380 that can be used with or without patch material 382 (see FIG. 21) in accordance with the present invention. First reinforcing portion 384 includes a plurality of eyelets 386 connected to eyelets 388 on second reinforcing portion 390 by tension member 392. The first and second anchor portions 384, 390 have a greater surface area of engagement with the tendon than the tension member 392, distributing loads over a greater area of tissue.

The tension member 392 forms a complete loop of material, the ends 394, 396 of which are connected by a knot 398. By pulling the ends 394, 396 of the tension member 392, the distance 400 between the first and second reinforcing portions 384, 390 can be reduced. Various loop structures and associated methods are disclosed in U.S. Pat. Nos. 7,090,111, 6,358,271, and 6,286,746, which are hereby incorporated by reference.

Since the tension member 392 is a complete loop, the system is fully equalized. That is, any load applied to the first and second ends 384, 390 is distributed along the full length of the tension member 392. Additionally, since the tension member 392 can slip within the eyelets 386, 388, the load direction 402 can be changed in either direction 404, 406, as illustrated in FIG. 20b or 20c. The tension member 392 is preferably constructed from a bioabsorbable material. Alternatively, the tension member 392 can be suture material, xenograft or allograft strips, or any of the other materials disclosed herein. In an alternate embodiment, the tension member 392 can be configured with a radially distributed load profile.

FIG. 21 illustrates the implant 380 used in combination with patch material 384. A pair of second reinforced ends 390a, 390b are engaged with the first reinforced end 384 using a pair of tension members 392a, 392b, respectively. The second reinforced ends 390a, 390b are free to move as illustrated in FIGS. 20a-20c, thereby shifting the load directions 402a, 402b. In an alternate embodiment, the second reinforced ends 390 can be located beyond the second edge 408 of the patch material 384.

In the illustrated embodiment, the patch material 384 optionally includes a plurality of pre-determined cut lines 384a. The cut lines 384a are welded or otherwise reinforced regions of the patch material 384 along which the surgeon can cut with minimal risk of fraying or unraveling.

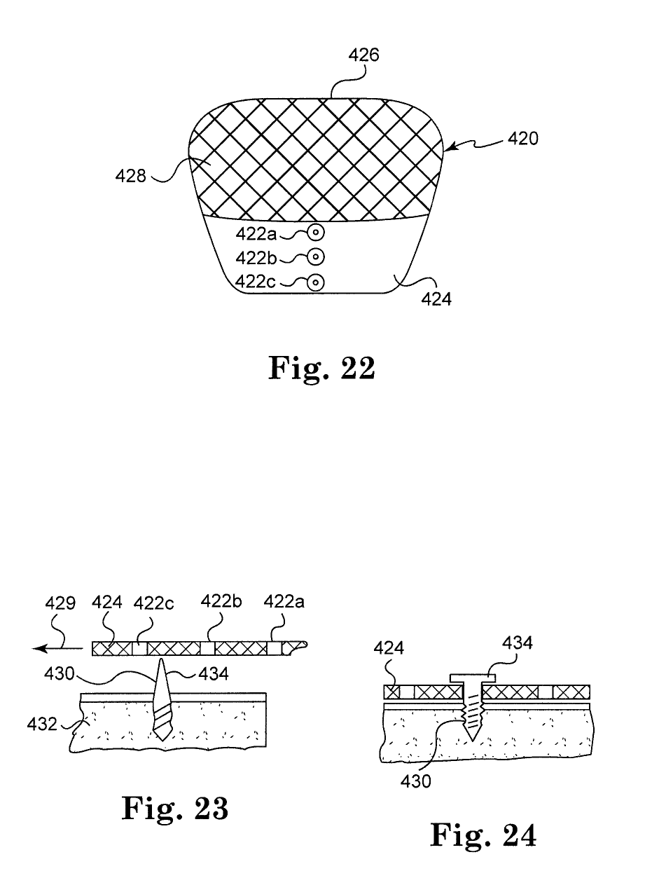

FIG. 22 illustrates an implant 420 with a plurality of eyelets 422a-422c (collectively “422”) formed in the first anchor portion 424. The second edge 426 of the patch material 428 is attached to the tendon using any of the methods disclosed herein. As best illustrated in FIG. 23, the surgeon applies a tension force 429 to the first end 424 and engages the appropriate eyelet 422a, 422b, 422c with bone anchors 430 previously affixed to the bone 432. As illustrated in FIG. 24, distal end 434 of the bone anchor 430 is then thermally or ultrasonically deformed to secure the first anchor portion 424. The shape of the distal end 434 forms an overlapping attachment with the first anchor portion 424. The bone anchor 430 retains the desired tension on the implant 420 while the surgeon provides additional attachment of the first end 424 using any of the methods disclosed herein. In an alternate embodiment, the tension members 332 illustrated in FIG. 19a may be used in combination with the implant 420.

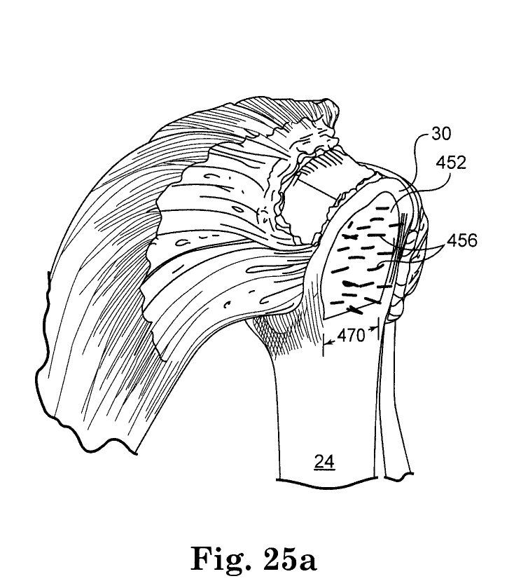

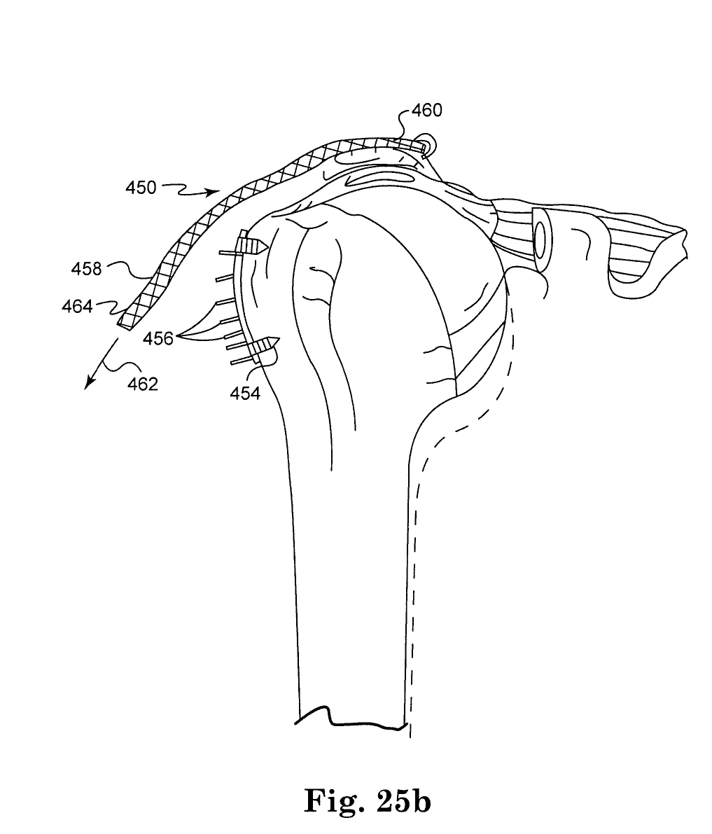

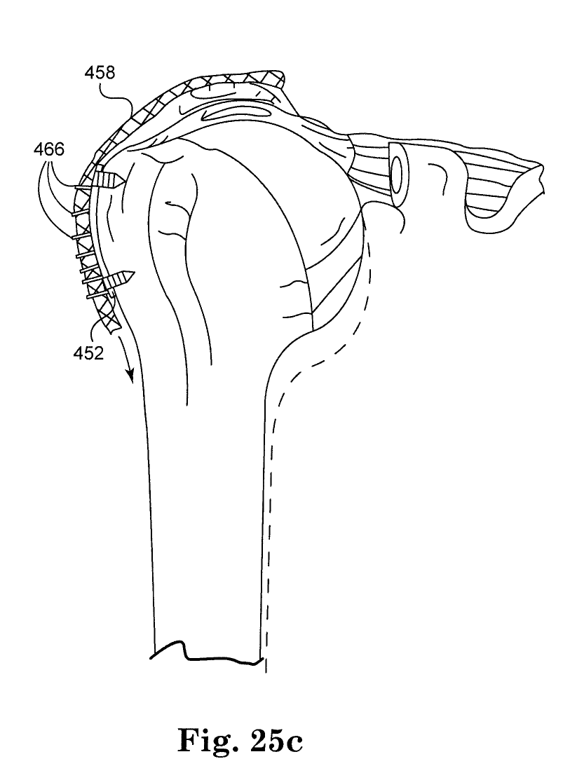

FIGS. 25a-25c illustrates an implant 450 that uses an anchor structure 452 attached to the greater tuberosity 30 of the humerus 24 in accordance with an embodiment of the present invention. The anchor structure 452 can be attached to the humerus using a variety of techniques, such as the bone anchors 454 illustrated in FIG. 25b. The anchor structure 452 includes a plurality of protrusions 456 adapted to engage and penetrate patch material 458.

As best illustrated in FIG. 25b, the surgeon attaches the medial edge 460 of the patch material 458 to the tendons. A tension force 462 is then applied to the lateral edge 464 of the patch material 458. As illustrated in FIG. 25c, the patch material 458 is engaged under tension with the protrusions 456 on the anchor structure 452. Distal ends 466 of the protrusions 456 are then thermally or ultrasonically deformed to affix the patch material 458 to the anchor structure 452. In an alternate embodiment, the protrusions 456 mechanically engage with the patch material 458, such as for example in the manner of a hook-and-loop fastener or a headed-stem fastener. The shape of the distal ends 466 and the anchor structure 452 form an overlapping attachment with the patch material 458.

The embodiment of FIGS. 25a-25c is particularly useful with a patch material 458 that is pre-formed with the hemispherical shape of the greater tuberosity 30. The width 470 of the anchor structure 452 permits the surgeon to create variable tension in the patch material 458 across the greater tuberosity 30.

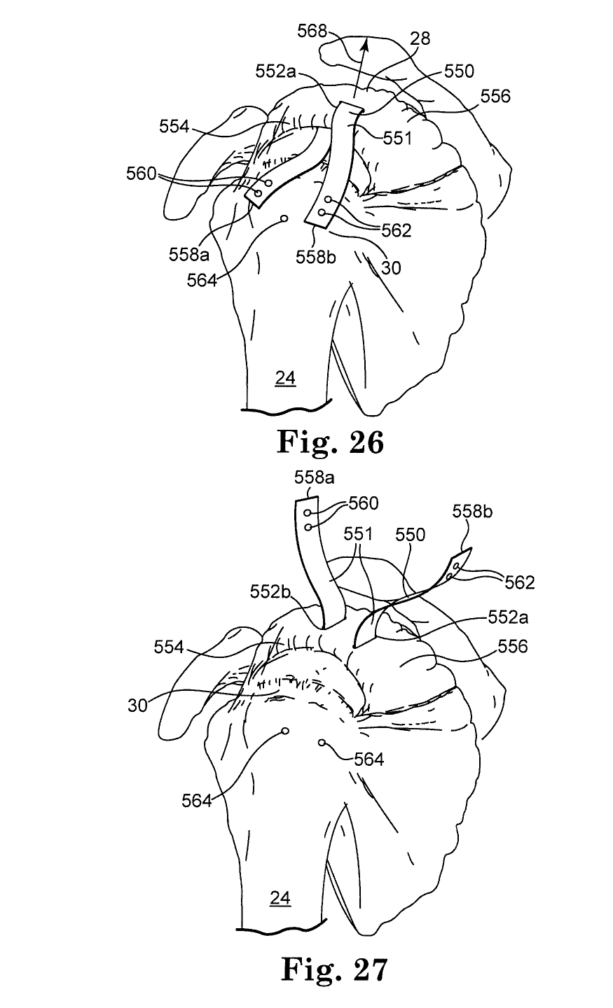

FIGS. 26 and 27 illustrate an alternate implant 550 threaded through one or more slits 552a, 552b in the medial portion 554 of the rotator cuff tendon 556. In the illustrated embodiment, first and second anchor portions 558a, 558b of the implant 550 include one or more eyelets 560, 562 adapted to engage with one or more attachment mechanisms, such as bone anchors 564, in the greater tuberosity 30 of the humerus 24. Tension member 551 of the implant 550 can be adjusted by selecting different eyelets 560, 562 and/or different bone anchors 564. As discussed above, the bone anchors 564 are optionally thermally or ultrasonically deformed to affix the implant 550.

In the embodiment of FIG. 26, the slit 552a is oriented perpendicular to the load direction 568. In the embodiment of FIG. 27, the slits 552a, 552b are oriented at about 45 degrees to the load direction 568. The first and second anchor portions 558a, 558b in FIG. 27 are optionally arranged in an X-pattern across the greater tuberosity 30.

In practice, once the implant 550 is attached to a bone anchor 564, additional mechanisms are optionally used to further secure the implant 550 to the bone. The implant 550 preferably include one or more pre-determined cut lines to facilitate removal of excess material. The embodiments of FIGS. 26 and 27 are optionally used with any of the patch materials disclosed herein. The patch material may be implanted either before or after the implant 550. The embodiments of FIGS. 14-17 and 20 are also particularly well suited to the methodology illustrated in FIGS. 26 and 27.

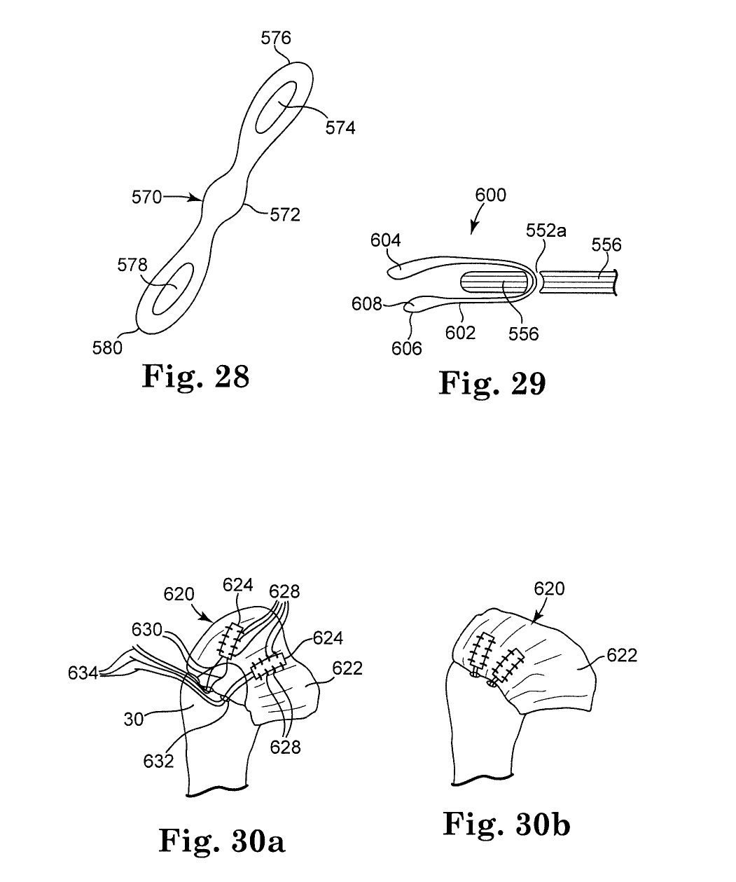

FIG. 28 illustrates an alternate implant 570 suitable for use in the procedure illustrated in FIGS. 26 and 27 in accordance with an alternate embodiment of the present invention. The implant 570 includes an elongated tension member 582 with loop 574 formed at first end 576. Middle portion 572 of the tension member 582 optionally includes an increased surface area of engagement to reduce the risk that the slits 552a, 552b in the tendon 556 will tear. Second end 580 optionally includes loop 578. In one embodiment, the implant 570 is threaded through one or more of the slits 552a, 552b in FIGS. 26 and 27. The loops 574, 578 can be attached to the greater tuberosity 30 using any of the techniques disclosed herein. In one preferred embodiment, the loops 574, 578 are attached to bone anchors 564 by suture material. The surgeon has the option to use the suture material to apply tension to the implant 570.

In another embodiment, the implant is threaded through one of the slits 552a, 552b and then the first end 576 is threaded through the loop 578 in the second end 580. This configuration cinches the implant 570 around the tendon 556. The first end 576 is then attached to the greater tuberosity 30 as discussed herein.

FIG. 29 is a side view of an alternate implant 600 also suitable for use in the procedure illustrated in FIGS. 26 and 27 in accordance with an alternate embodiment of the present invention. The implant 600 is a continuous loop 602 folded in half to form first end 604 and second end 606. One of the ends 604, 606 is threaded through one of the slits 552a, 552b as illustrated in FIG. 29. In one embodiment, the first and second ends 604, 606 are attached to the greater tuberosity 30 as discussed above.

In an alternate embodiment, first end 604 is inserted through loop 608 formed in the second end 606. This configuration girth hitches the implant 600 to the tendon 556. The first end 604 is then attached to the greater tuberosity 30 using any of the techniques disclosed herein.

FIG. 30a illustrates a shoulder 620 with a retracted tear in the rotator cuff 622. Patch material 624 on implant 626 is attached to the rotator cuff 622 using a plurality of sutures 628. The size and shape of the patch material 624 plus the plurality of sutures 628 operate to distribute tension loads over a larger area of the tissue 622.

The patch material 624 includes one or more tension members 630. The tension members 630 preferably include an overlapping attachment to the patch material 624, and are preferably pre-attached by the manufacturer. The tension members 630 are attached to the greater tuberosity 30 using a variety of techniques. In the illustrated embodiment, the tension members 630 are cinched to bone anchors 632. Distal ends 634 of the tension members 630 permit the surgeon to apply tension to the implant 626. Once the desired level of tension is achieved, the surgeon ties-off the tension members 630 on the bone anchors 632. FIG. 30b illustrates the shoulder 620 with the rotator cuff 622 tensioned into an anatomically correct location. Distal ends 634 of the tension members 630 have been removed.

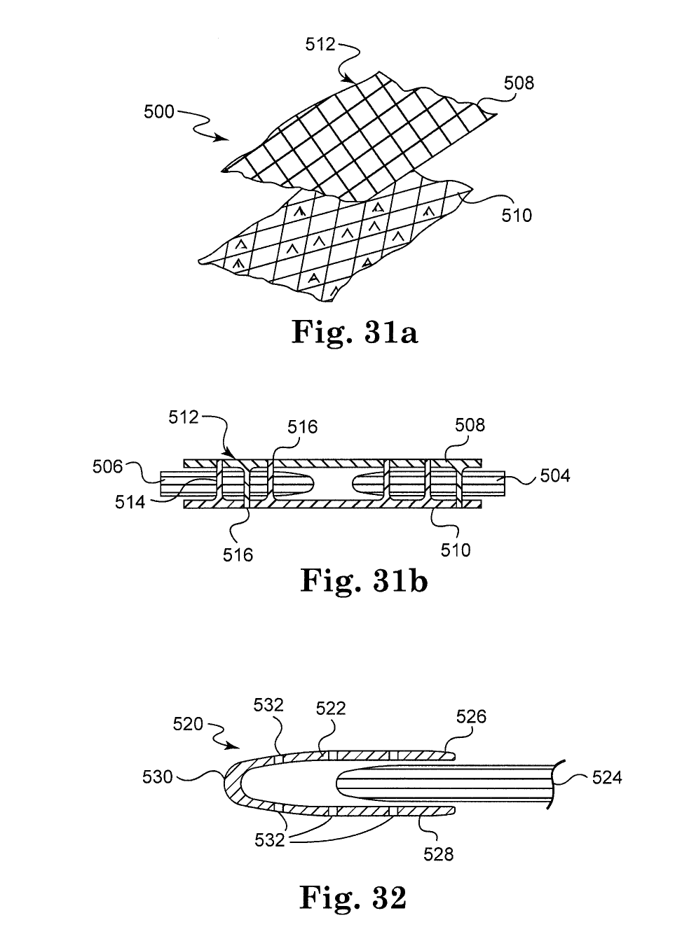

FIGS. 31a and 31b illustrate an alternate method and apparatus for attaching an implant 500 to tissue or patch material in accordance with an embodiment of the present invention. The members 504, 506 are compressed between upper portion 508 and lower portion 510 of the anchor portion 512. One or both of the upper and lower portions 508, 510 include a plurality of protrusions 514 that penetrate the members 504 and 506 through to the opposite portion 508, 510.

In one embodiment, distal ends 516 of the protrusions 514 are deformed using ultrasonic or thermal energy, thereby capturing the members 504 and 506. In an alternate embodiment, only the lower portion 510 includes the protrusions 514. In one embodiment, member 504 is the medial portion of the tendon and member 506 is the patch material. In an alternate embodiment, member 504 is the medial portion of the tendon and member 506 is the lateral tendon. In another embodiment, the protrusions 514 mechanically engage with the upper and lower portions 508, 510, such as in the manner of a hook-and-loop fastener or headed-stem fastener.

FIG. 32 illustrate an alternate method and apparatus for attaching an implant 520 to native tissue in accordance with an embodiment of the present invention. The patch material 522 is folded around the medial portion of the tendon 524. The distal edges 526, 528 are attached to the medial portion of the tendon 524 using any of the techniques disclosed herein. In this way, tension loads are distributed over a large area of tendon 524 and the risk of suture pull-out is reduced. The folded edge 530 is then fastened to the bone in the vicinity of the lateral edge of the tendon. The patch material 522 optionally includes a plurality of openings 532 to promote tissue in-growth.

### Biological Agents

In certain embodiments of the present invention, the implant may be coated with biologically active agents. These agents may include natural or synthetic heparin binding growth factors (HBGFs) that are useful as biologically active agents for coating of medical devices, such as for instance, sutures, implants and medical instruments to promote biological responses, for instance, to stimulate growth and proliferation of cells, or healing of wounds. Representative HBGFs include, for example, known FGFs (FGF-1 to FGF-23), HBBM (Heparin-binding brain mitogen), HB-GAF (heparin-binding growth associated factor), HB-EGF (heparin-binding EGF-like factor) HB-GAM (heparin-binding growth associated molecule, also known as pleiotrophin, PTN, HARP), TGF-.alpha. (transforming growth factor-.alpha.), TGF-.beta.s (transforming growth factor-.beta.s), VEGF (vascular endothelial growth factor), EGF (epidermal growth factor), IGF-1 (insulin-like growth factor-1), IGF-2 (insulin-like growth factor-2), PDGF (platelet derived growth factor), RANTES, SDF-1, secreted frizzled-related protein-1 (SFRP-1), small inducible cytokine A3 (SCYA3), inducible cytokine subfamily A member 20 (SCYA20), inducible cytokine subfamily B member 14 (SCYB 14), inducible cytokine subfamily D member 1 (SCYD1), stromal cell-derived factor-1 (SDF-1), thrombospondins 1, 2, 3 and 4 (THBS1 4), platelet factor 4 (PF4), lens epithelium-derived growth factor (LEDGF), midikine (MK), macrophage inflammatory protein (MIP-1), moesin (MSN), hepatocyte growth factor (HGF, also called SF), placental growth factor, IL-1 (interleukin-1), IL-2 (interleukin-2), IL-3 (interleukin-3), IL-6 (interleukin-6), IL-7 (interleukin-7), IL-10 (interleukin-10), IL-12 (interleukin-12), IFN-.alpha. (interferon-.alpha.), IFN-.gamma. (interferon-.gamma.), TNF-.alpha. (tumor necrosis factor-.alpha.), SDGF (Schwannoma-derived growth factor), nerve growth factor, neurite growth-promoting factor 2 (NEGF2), neurotrophin, BMP-2 (bone morphogenic protein 2), OP-1 (osteogenic protein 1, also called BMP-7), keratinocyte growth factor (KGF), interferon-y inducible protein-20, RANTES, and HIV-tat-transactivating factor, amphiregulin (AREG), angio-associated migratory cell protein (AAMP), angiostatin, betacellulin (BTC), connective tissue growth factor (CTGF), cysteine-rich angiogenic inducer 61 (CYCR61), endostatin, fractalkine/neuroactin, or glial derived neurotrophic factor (GDNF), GRO2, hepatoma-derived growth factor (HDGF), granulocyte-macrophage colony stimulating factor (GMCSF), and the many growth factors, cytokines, interleukins and chemokines that have an affinity for heparin.

Surfaces suitable for biological coatings may be formed from any of the commonly used materials suitable for use in medical devices, such as for instance, stainless steel, titanium, platinum, tungsten, ceramics, polyurethane, polytetrafluoroethylene, expanded polytetrafluoroethylene, polycarbonate, polyester, polypropylene, polyethylene, polystyrene, polyvinyl chloride, polyamide, polyacrylate, polyurethane, polyvinyl alcohol, polycaprolactone, polylactide, polyglycolide, polydioxanone, trimethylene carbonate, polysiloxanes (such as 2,4,6,8-tetramethylcyclotetrasiloxane), polyhydroxyalkanoates such as poly 4-hydroxybutyrate, silk, collagen, allogeneic or xenogeneic tissues, natural rubbers, or artificial rubbers, or blends, block polymers or copolymers thereof.

Methods for coating biological molecules onto the surfaces of medical devices are known. See for instance U.S. Pat. No. 5,866,113 to Hendriks et al., the specification of which is hereby incorporated by reference. Tsang et al. in U.S. Pat. No. 5,955,588 teach a non-thrombogenic coating composition and methods for using the same on medical devices, and is incorporated herein by reference. Zamora et al in U.S. Pat. No. 6,342,591 teach an amphipathic coating for medical devices for modulating cellular adhesion composition, and is incorporated herein by reference.

In some embodiments the implant of this invention may be coated with a synthetic HGBF analog.

Suitable synthetic HGBF analogs are also represented be an agent of formula I or formula II. The regions X, Y and Z of the synthetic HBGF analogs of formula I or formula II include amino acid residues. An amino acid residue is defined as —NHRCO—, where R can be hydrogen or any organic group. The amino acids can be D-amino acids or L-amino acids. Additionally, the amino acids can be .sigma.-amino acids, .beta.-amino acids, .gamma.-amino acids, or .delta.-amino acids and so on, depending on the length of the carbon chain of the amino acid.

The amino acids of the X, Y and Z component regions of the synthetic HBGF analogs of the invention can include any of the twenty amino acids found naturally in proteins, i.e. alanine (ala, A), arginine (Arg, R), asparagine (Asn, N), aspartic acid (Asp, D), cysteine (Cys, C), glutamic acid (Glu, E), glutamine (Gln, Q), glycine (Gly, G), histidine (His, H), isoleucine, (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), and valine (Val, V).

Furthermore, the amino acids of the X, Y and Z component regions of these synthetic HBGF analogs may include any of the naturally occurring amino acids not found naturally in proteins, e.g. beta.-alanine, betaine (N,N,N-trimethylglycine), homoserine, homocysteine, .gamma.-amino butyric acid, ornithine, and citrulline.

Additionally, the amino acids of the X, Y and Z component regions of these synthetic HBGF analogs may include any of the non-biological amino acids, i.e. those not normally found in living systems, such as for instance, a straight chain amino-carboxylic acid not found in nature. Examples of straight chain amino-carboxylic acids not found in nature include 6-aminohexanoic acid, and 7-aminoheptanoic acid, 9-aminononanoic acid and the like.

In formula I when n is 0, the synthetic analogs include a single X region and the molecule is a linear chain. When n is 1 in formula I, the molecule includes two X regions that are identical in amino acid sequence. In the latter case the molecule is a branched chain that may also be constrained by cross-links between the two X regions as described below. In this embodiment, the HBGF analog may bind two HBGFRs and induce receptor dimerization. Advantageously, the dimerization in turn potentiates enhanced receptor signaling activity of the HBGFRs.

When n is 0 in formula I, the X region of the synthetic HBGF analog is covalently linked through an amino acid, J.sub.1 to the hydrophobic region Y.

When n is 1 in formula I, one X region is covalently linked through an amino acid J.sub.1, which is in turn covalently linked to a second amino acid, J.sub.2, which is a diamino acid. J.sub.1 is linked to one amino group of the diamino acid, J.sub.2. The second X region is covalently linked to J.sub.2 through the second amino group of the diamino acid. J.sub.2 is then covalently linked through its carboxy terminus to the Y region of the synthetic HBGF analog.

The amino acid J.sub.1 of formula I can be any of the amino acids described above. The diamino acid J.sub.2 of formula I can be any diamino acid, such as for instance lysine, or ornithine, or any other amino acid having two amino groups.

The region, X of formula I of the synthetic these HBGF analogs is a synthetic peptide chain that binds an HBGF receptor (HBGFR). Region X can, for example, have any amino acid sequence that binds an HBGFR, and can include amino acid sequences that are identical to a portion of the amino acid sequence of a HBGF. Alternatively, X can have an amino acid sequence homologous rather than identical to the amino acid sequence of an HBGF. The particular HBGFR bound by the synthetic HBGF analog may or may not be the cognate receptor of the original HBGF, i.e. the synthetic HBGF analog may additionally or solely bind to the receptor of a different HBGF.

The term ‘homologous’, as used herein refers to peptides that differ in amino acid sequence at one or more amino acid positions when the sequences are aligned. For example, the amino acid sequences of two homologous peptides can differ only by one amino acid residue within the aligned amino acid sequences of five to ten amino acids. Alternatively, two homologous peptides of ten to fifteen amino acids can differ by no more than two amino acid residues when aligned. In another alternative, two homologous peptides of fifteen to twenty or more amino acids can differ by up to three amino acid residues when aligned. For longer peptides, homologous peptides can differ by up to approximately 5%, 10%, 20% or 25% of the amino acid residues when the amino acid sequences of the two peptide homologs are aligned.

Suitable amino acid sequences as X regions of formula I include homologs of fragments of naturally occurring HBGFs that differ from the amino acid sequences of natural growth factor in only one or two or a very few positions. Such sequences preferably include conservative changes, where the original amino acid is replaced with an amino acid of a similar character according to well known principles; for example, the replacement of a non-polar amino acid such as alanine with valine, leucine, isoleucine or proline; or the substitution of one acidic or basic amino acid with another of the same acidic or basic character.

In another alternative, the X region of the synthetic HBGF analog can include an amino acid sequence that shows no detectable homology to the amino acid sequence of any HBGF. Peptides or growth factor analogs useful as components of the X region of the synthetic analogs of the present invention, that have little or no amino acid sequence homology with the cognate growth factor and yet bind HBGFRs may be obtained by any of a wide range of methods, including for instance, selection by phage display. See as an example: Sidhu et al. Phage display for selection of novel binding peptides. Methods Enzymol 2000; vol. 328:333 63.

The X region of the synthetic HBGF analogs may have any length that includes an amino acid sequence that effectively binds an HBGFR. Preferably, the synthetic HBGF analogs have a minimum length of at least approximately three amino acid residues. Some synthetic HBGF analogs have a minimum length of at least approximately six amino acid residues. Other synthetic HBGF analogs have a minimum length of at least approximately ten amino acid residues. The synthetic HBGF analogs may also have a maximum length of up to approximately fifty amino acid residues. Some synthetic HBGF analogs have a maximum length of up to approximately forty amino acid residues. Other synthetic HBGF analogs have a maximum length of up to approximately thirty amino acid residues.

In another embodiment of the synthetic HBGF analogs that include two X regions, the X regions are covalently cross linked. Suitable cross links can be formed by S—S bridges of cysteines linking the two X regions. Alternatively, the cross link can be conveniently formed during simultaneous and parallel peptide synthesis of the X region amino acids chains by incorporating a lanthionine (thio-dialanine) residue to link the two identical X chains at alanine residues that are covalently bonded together by a thioether bond. In another method the two X region amino acid chains can be cross-linked by introducing a cross-linking agent, such as a dicarboxylic acid, e.g. suberic acid (octanedioic acid), or the like, thereby introducing a hydrocarbon bridge between the two identical X regions having a free amino, hydroxyl or thiol group.

In the synthetic HBGF analogs, the Y region of formula I represents a linker that is sufficiently hydrophobic to non-covalently bind the HBGF analog to a polystyrene or polycaprolactone surface, or the like. In addition, the Y region may bind to other hydrophobic surfaces, particularly the hydrophobic surfaces formed from materials used in medical devices. Such surfaces are typically hydrophobic surfaces. Examples of suitable surfaces include but are not limited to those formed from hydrophobic polymers such as polycarbonate, polyester, polypropylene, polyethylene, polystyrene, polytetrafluoroethylene, expanded polytetrafluoroethylene, polyvinyl chloride, polyamide, polyacrylate, polyurethane, polyvinyl alcohol, polyurethane, poly ethyl vinyl acetate, poly(butyl methacrylate), poly(ethylene-co-vinyl acetate), polycaprolactone, polylactide, polyglycolide, PDS, TMC, PHA's, and copolymers of any two or more of the foregoing; siloxanes such as 2,4,6,8-tetramethylcyclotetrasiloxane; natural and artificial rubbers; glass; biological materials, and metals including stainless steel, titanium, platinum, and nitinol.

The Y region of formula I includes a chain of atoms or a combination of atoms that form a chain. Typically, the chains are chains of carbon atoms, that may also optionally include oxygen, nitrogen or sulfur atoms, such as for example chains of atoms formed from amino acids (e.g. amino acids found in proteins, as listed above; naturally occurring amino acids not found in proteins, such as ornithine and citrulline; or non natural amino acids, such as amino hexanoic acid; or a combination of any of the foregoing amino acids).

The chain of atoms of the Y region of formula I is covalently attached to J.sub.1 or J.sub.2, and to peptide Z. The covalent bonds can be, for example, amide or ester bonds.

This Y region includes a chain of a minimum of about nine atoms. In some embodiments, the Y region includes a chain of a minimum of about twelve atoms. In other embodiments, the Y region includes a chain of a minimum of about fifteen atoms. For example, the Y region may be formed from a chain of at least four, at least five or at least six amino acids. Alternatively, the Y region may be formed from a chain of at least one, at least two, or at least three aminohexanoic acid residues.

In suitable embodiments, the Y region includes a chain of a maximum of about fifty atoms. IN some embodiments, the Y region includes a chain of a maximum of about forty-five atoms. In other embodiments, the Y region includes a chain of a maximum of about thirty-five atoms. For example, the Y region may be formed from a chain of up to about twelve, up to about fifteen, or up to about seventeen amino acids.

The amino acid sequence of the Y region of formula I is an artificial sequence, i.e. it does not include any amino acid sequence of four or more amino acid residues found in a natural ligand of a HBGF.

In a particular embodiment, the Y region includes a hydrophobic amino acid residue, or a chain of hydrophobic amino acid residues. The Y region may, for example, include one or more aminohexanoic acid residues, such as one, two, three or more aminohexanoic acid residues.

In another particular embodiment, the Y region of the molecule of formula I may include a branched or unbranched, saturated or unsaturated alkyl chain of between one and about twenty carbon atoms. In a further embodiment, the Y region may include a chain of hydrophobic residues, such as for instance, ethylene glycol residues. For example, the Y region may include at least about three, or at least about four, or at least about five ethylene glycol residues. Alternatively, the Y region may include up to about twelve, up to about fifteen, or up to about seventeen ethylene glycol residues.

In another alternative embodiment, the Y region may include a combination of amino acid and hydrophobic residues.

The hydrophobic Y region of these HBGF analogs is covalently linked to the Z region.

The Z region of the analog formula I is a heparin-binding region and can include one or more heparin-binding motifs, BBxB or BBBxxB as described by Verrecchio et al. J. Biol. Chem. 275: 7701, (2000). Alternatively, the Z region may include both BBxB and BBBxxB motifs (where B represents lysine, arginine, or histidine, and x represents a naturally occurring, or a non-naturally occurring amino acid). For example, the heparin-binding motifs may be represented by the sequence [KR][KR][KR]X(2)[KR], designating the first three amino acids as each independently selected from lysine or arginine, followed by any two amino acids and a sixth amino acid which is lysine or arginine.

The number of heparin binding motifs is not critical. For instance, the Z region may include at least one, at least two, at least three or at least five heparin-binding motifs. Alternatively, the Z region may include up to a maximum of about ten heparin-binding motifs. In another alternative embodiment, the Z region includes at least four, at least six or at least eight amino acid residues. Further, the Z region may include up to about twenty, up to about, twenty-five, or up to about thirty amino acid residues.

In a preferred embodiment, the amino acid sequence of the Z region is RKRKLERIAR. Heparin-binding domains that bear little or no sequence homology to known heparin-binding domains are also suitable. As used herein the term “heparin-binding” means binding to the —NHSO.sub.3.sup.- and sulfate modified polysaccharide, heparin and also binding to the related modified polysaccharide, heparan.

The Z region of the synthetic HBGF analogs confers the property of binding to heparin in low salt concentrations, up to about 0.48M NaCl, forming a complex between heparin and the Z region of the factor analog. The complex can be dissociated in 1M NaCl to release the synthetic HBGF analog from the heparin complex.

The Z region is a non-signaling peptide. Accordingly, when used alone the Z region binds to heparin which can be bound to a receptor of a HBGF, but the binding of the Z region peptide alone does not initiate or block signaling by the receptor.

The C-terminus of the Z region may be blocked or free. For example, the C terminus of the Z region may be the free carboxyl group of the terminal amino acid, or alternatively, the C terminus of the Z region may be a blocked carboxyl group, such as for instance, an amide group. In a preferred embodiment the C terminus of the Z region is an amidated arginine.

In another embodiment, the HBGF synthetic analog is an agent represented by formula II. The synthetic HFGF analog represented by formula II is an analog of an fibroblast growth factor (FGF) which can be any FGF, such as any of the known FGFs, including all 23 FGFs from FGF-1 to FGF-23.

The X region of the agent of formula II may include an amino acid sequence found in an FGF, such as for instance, FGF-2 or FGF-7. Alternatively, the X region can include a sequence not found in the natural ligand of the FGFR bound by the agent of formula II.

The F and Z regions of formula II are subject to the same limitations in size and sequence as described above for the corresponding X and Z regions of formula I.

The Y region of the HBGF analogs of formula II have the same size limitations as the Y region of the HBGF analogs of formula I. However, the overall physical characteristics of the Y region of formula II is not limited to hydrophobic properties and can be more varied. For example, the Y region of formula II can be polar, basic, acidic, hydrophilic or hydrophobic. Thus, the amino acid residues of the Y region of formula II can include any amino acid, or polar, ionic, hydrophobic or hydrophilic group.

The X region of the synthetic HBGF of formula II can include an amino acid sequence that is 100% identical to the amino acid sequence found in a fibroblast growth factor or an amino acid sequence homologous to the amino acid sequence of a fibroblast growth factor. For instance, the X region can include an amino acid sequence that is at least about 50%, at least about 75%, or at least about 90% homologous to an amino acid sequence from a fibroblast growth factor. The fibroblast growth factor can be any fibroblast growth factor, including any of the known or yet to be identified fibroblast growth factors.

In a particular embodiment, the synthetic FGF analog of the invention is an agonist of the HBGFR. When bound to the HBGFR, the synthetic HBGF analog initiates a signal by the HBGFR.

In a further particular embodiment, the synthetic FGF analog of the invention is an antagonist of the HBGFR. When bound to the HBGFR, the synthetic HBGF analog blocks signaling by the HBGFR.

In another particular embodiment of the present invention, the synthetic FGF analog is an analog of FGF-2 (also known as basic FGF, or bFGF). In another particular embodiment of the present invention, the binding of the synthetic FGF analog to an FGF receptor initiates a signal by the FGF receptor. In a further particular embodiment, the binding of the synthetic FGF analog to the FGF receptor blocks signaling by the FGF receptor.

In a yet further particular embodiment, the present invention provides a synthetic FGF analog of FGF-2, wherein the FGF receptor-binding domain is coupled through a hydrophobic linker to a heparin-binding domain. In another particular embodiment, the present invention provides a synthetic FGF analog of FGF-2, wherein the amino acid sequence of the F region is YRSRKYSSWYVALKR from FGF-2. In yet another particular embodiment, the synthetic FGF analog has the amino acid sequence NRFHSWDCIKTWASDTFVLVCYDDGSEA in the F region.

Specific HGBF analogs and process for making these analogs are reported in U.S. Pat. No. 7,166,574 which is incorporated herein by reference.

Patents and patent applications disclosed herein, including those cited in the Background of the Invention, are hereby incorporated by reference. Other embodiments of the invention are possible, including by recombining the various elements disclosed herein. Although the description above contains many specificities, these should not be construed as limiting the scope of the invention, but as merely providing illustrations of some of the presently preferred embodiments of this invention. Thus the scope of this invention should be determined by the appended claims and their legal equivalents. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims.

What is claimed is:

1. An implant for the repair of a tendon or a ligament along at least one load direction, the implant comprising:
at least one first anchor portion; and
at least one tension member adapted to be oriented along a load direction and secured to the first anchor portion with an overlapping attachment.

2. The implant of claim 1 wherein the first anchor portion comprises a first surface area of engagement and the tension member comprises a second surface area of engagement less than the first surface area of engagement.

3. The implant of claim 1 comprising a second anchor portion attached to the tension member offset from the first anchor portion, wherein the tension member comprises the sole portion of the implant located in a center region located between the first anchor portion and the second anchor portion.

4. The implant of claim 1 comprising a second anchor portion attached to the tension member and offset from the first anchor portion.

5. The implant of claim 1 comprising:
a second anchor portion attached to the tension member offset from the first anchor portion; and
a patch material bordering the center region.

6. The implant of claim 1 comprising a bioabsorbable patch material extending from at least the first anchor portion to a distal end of the tension member.

7. The implant of claim 6 wherein the tension members are secured to a patch material at a location between the first anchor portion and the distal end of the tension member.

8. The implant of claim 1 wherein at least one of the first anchor portion and the tension member comprise a scalable weave.

9. The implant of claim 1 comprising:
at least one bone anchor; and
a plurality of eyelets in the first anchor portion aligned along a load direction adapted to engage with the bone anchor to permit adjustment of tension in the implant.

10. The implant of claim 1 wherein the first anchor portion comprises:
a first layer with a plurality of protrusions adapted to penetrate the tendon or ligament on one surface; and
a second layer adapted to engage with distal ends of the protrusions on the other side of the tendon or ligament.

11. The implant of claim 1 wherein one or more of the first anchor portion and the tension member comprises a bioabsorbable material with a strength retention after implantation of about 50% after about 2 months to about 50% after about 6 months.

12. The implant of claim 1 comprising a plurality of discrete tension members oriented along a plurality of load directions.

13. The implant of claim 1 wherein the tension member comprises a plurality of tension members with a radially distributed load profile corresponding generally to a plurality of load directions.

14. The implant of claim 1 wherein at least one of the tension member and the first anchor portion comprises a pre-determined cut line.

15. The implant of claim 1 wherein the tension member comprises an enlarged middle portion adapted to engage with a slit in the tendon or ligament.

16. The implant of claim 1 comprising at least one discrete tension member attachable to the first anchor portion.

17. The implant of claim 1 comprising:
a first bone anchor engaged with the first anchor portion; and
a first tension member comprising a first end with at least one eyelet pivotally engaged with the first bone anchor along a first load direction in an overlapping attachment.

18. The implant of claim 17 comprising a second tension member with a reinforced second end and at least one eyelet engaged with the first bone anchor along a second load direction.

19. The implant of claim 1 comprising:
at least one elongated slot in a patch material located between the first anchor portion and a second anchor portion offset from the first anchor portion; and
a bioabsorbable suture material laced in edges of the elongated slot, wherein tension on the suture material reduces the elongated slot to increase tension between the first anchor portion and the second anchor portion along a load direction.

20. The implant of claim 19 comprising at least one elongated slot comprising a primary axis oriented perpendicular to a load direction.

21. The implant of claim 1 comprising:
a plurality of discrete second anchor portions offset from the first anchor portion; and
at least one tension member connecting each of the second anchor portions to the first anchor portion.

22. The implant of claim 1 wherein the first anchor portion comprises a plurality of protrusions adapted to mechanically engage with a patch material.

23. The implant of claim 1 wherein the first anchor portion comprises:
a first layer with a plurality of protrusions adapted to penetrate the tendon or ligament on one surface; and
a second layer adapted to engage with distal ends of the protrusions on the other side of the tendon or ligament.

24. The implant of claim 1 comprising a rotator cuff repair implant.

25. The implant of claim 1 wherein at least one of the first anchor portion and the tension member comprise polyhydroxyalkanoate fibers.

26. The implant of claim 1 comprising sutures, tacks, bone anchors, glue, staples, and combinations thereof adapted to affix the implant to tendon, ligament or bone.

27. The implant of claim 1 wherein the implant comprises an active agent selected from the group consisting of therapeutic, diagnostic, and prophylactic agents.

28. The implant of claim 1 comprising a growth factor.

29. The implant of claim 1 wherein one or more of the first anchor portion or the tension member is selected from the group consisting of auto graft, allograft, and xenograft.

30. The implant of claim 1 wherein one or more of the first anchor portion and the tension member comprise one or more layers of a non-woven mesh, a knitted, woven or braided multifilament and/or monofilament mesh, a multi-component structure, a scalable weave, terrycloth structure, film, or a combination thereof.

31. The implant of claim 1 wherein the tension member comprises at least one non-bioabsorbable reinforcing fibers.

32. An implant for the repair of a tendon or a ligament along at least one load direction, the implant comprising:
at least one first anchor portion;
at least one second anchor portion offset from the first anchor portion;
at least one tension member oriented along a load direction and connecting the first and second anchor portions, the tension member connected to at least one of the first or second anchor portions with an overlapping attachment; and
a center region in the offset between the first and second anchor portions, wherein the tension member comprises more than 50% of the material located in the center region.

33. An implant for the repair of a tendon or a ligament along at least one load direction, the implant comprising:
at least one first anchor portion;
at least one second anchor portion offset from the first anchor portion; and
a plurality of tension members comprising a radially distributed load profile corresponding generally to a plurality of load directions.

34. The implant of claim 33 comprising a center region in the offset between the first anchor portion and the second anchor portion, wherein the tension members comprise more than 50% of the material located in the center region.

35. An implant for the repair of a tendon or a ligament along at least one load direction, the implant comprising:
at least one first anchor portion;
at least one second anchor portion offset from the first anchor portion; and
at least one tension member comprising an elongated member laced through eyelets in the first anchor portion and the second anchor portion in a continuous loop.

36. The implant of claim 35 wherein the tension members comprises an equalized structure.

37. An implant for the repair of a tendon or a ligament along at least one load direction, the implant comprising:
at least one first anchor portion;
at least one second anchor portion offset from the first anchor portion;
a first bone anchor engaged with the first anchor portion; and
a first discrete tension member oriented along a first load direction comprising a first end adapted to engage with the first bone anchor.

38. The implant of claim 37 wherein the first tension member is rotatably engaged with the first bone anchor.

39. The implant of claim 37 comprising a second tension member with a reinforced first end adapted to engage with the first bone anchor along a second load direction.

40. An implant for the repair of a tendon or a ligament along at least one load direction, the implant comprising:
a patch material comprising a first edge and a second edge;
at least one elongated slot in the patch material in a location between the first and second edges; and
a suture material laced along opposite edges of the elongated slot, wherein tension on the suture material reduces the elongated slot to increase tension between the first and second edges along a load direction.

41. The implant of claim 40 comprising a plurality of elongated slots each comprising a primary axis oriented perpendicular to a load direction.

42. An implant for the repair of a tendon or a ligament along at least one load direction, the implant comprising:
a first layer comprising a plurality of protrusions adapted to penetrate a tendon or a ligament;
a second layer adapted to engage with distal ends of the protrusions on the other side of the tendon or ligament;
at least one first anchor portion offset from the first and second layers; and
a tension member connecting the first and second layers to a first anchor portion.

43. An implant for the repair of a tendon or a ligament along at least one load direction, the implant comprising:
at least one first anchor portion;
at least one second anchor portion offset from the first anchor portion;
at least one tension member oriented along a load direction and connecting the first anchor portion to the second anchor portion; and
at least one tension adjusting device adapted to adjust tension on the tension member.

44. The implant of claim 43 wherein the tension adjusting device comprises one or more holding devices that adjustably engage with the tension members to vary the offset from the first anchor portion to the second anchor portion.

45. The implant of claim 43 wherein the tension adjusting device comprises a plurality of eyelets in the first anchor portion aligned along the load direction adapted to selectively engage with bone anchors.

46. The implant of claim 43 wherein the tension member comprises an elongated member laced through eyelets in the first anchor portion and the second anchor portion in a continuous loop, wherein the tension adjusting device comprises adjustment to the length of the continuous loop.

47. The implant of claim 43 wherein the tension adjusting device comprises:
a patch material extending from at least the first anchor portion to the second anchor portion;
at least one elongated slot in a patch material; and
at least one suture material laced along opposite edges of the elongated slot, wherein tension on the suture material reduces the elongated slot to increase tension between the first anchor portion and the second anchor portion along a load direction.

48. An implant for the repair of a tendon or a ligament along at least one load direction, the implant comprising a patch material of a scalable weave.

49. An implant for the repair of a tendon or a ligament along at least one load direction, the implant comprising a patch material with at least one pre-determined cut line.

50. A method of repairing a tendon or a ligament comprising the steps of:
attaching at least one first anchor portion to tendon, ligament or bone;
orienting at least one tension member secured to the first anchor portion with an overlapping attachment along a load direction; and
attaching distal ends of the tension member to tendon, ligament or bone.

51. The method of claim 50 wherein the first anchor portion comprises a first surface area of engagement and the tension member comprises a second surface area of engagement less than the first surface area of engagement.

52. The method of claim 50 comprising attaching a second anchor portion to the tension member offset from the first anchor portion, wherein the tension member comprising the sole portion of the implant located in a center region located between the first anchor portion and the second anchor portion.

53. The method of claim 50 comprising the steps of:
attaching a second anchor portion to the tension member offset from the first anchor portion; and
attaching a patch material bordering a center region located in the offset between the first anchor portion and the second anchor portion.

54. The method of claim 50 comprising constructing at least one of the first anchor portion and the tension member from a scalable weave.

55. The method of claim 50 comprising:
implanting at least one bone anchor in bone; and
adjusting tension in the implant by engaging one of a plurality of eyelets in the first anchor portion aligned along a load direction with the bone anchor.

56. The method of claim 50 comprising the steps of:
inserting a plurality of protrusions in a first layer of the first anchor portion through the tendon or ligament; and
engaging a second layer of the first anchor portion with the distal ends of the protrusions on the other side of the tendon or ligament.

57. The method of claim 50 comprising selecting one or more of the first anchor portion and the tension member from a bioabsorbable material with a strength retention after implantation of about 50% after about 2 months to about 50% after about 6 months.

58. The method of claim 50 comprising orienting a plurality of discrete tension members along a plurality of load directions.

59. The method of claim 50 comprising orienting a plurality of tension members in a radially distributed load profile corresponding generally to a plurality of load directions.

60. The method of claim 50 comprising cutting at least one of the tension member and the first anchor portion along a pre-determined cut line.

61. The method of claim 50 comprising:
attaching a first bone anchor to a bone;
engaging a first end of a first tension member with the first bone anchor along a first load direction; and
engaging a first end of a second tension member with the first bone anchor along a second load direction.

62. The method of claim 50 comprising:
locating at least one elongated slot in a patch material located between the first anchor portion and distal ends of the tension member;
lacing a bioabsorbable suture material in edges of the elongated slot; and
applying tension to the suture material to reduce the elongated slot and to increase tension between the first anchor portion and the distal ends of the tension member.

63. The method of claim 50 comprising a rotator cuff repair.

64. The method of claim 50 comprising constructing the implant from a bioabsorbable material.

65. The method of claim 50 comprising applying an active agent to the implant selected from the group consisting of therapeutic, diagnostic, and prophylactic agents.

66. The method of claim 50 comprising constructing one or more of the first anchor portion or the tension member from the group consisting of auto graft, allograft, and xenograft.

67. The method of claim 50 comprising constructing one or more of the first anchor portion and the tension member from one or more layers of a non-woven mesh, a knitted, woven or braided multifilament and/or monofilament mesh, a multi-component structure, a scalable weave, terrycloth structure, film, or a combination thereof.

68. The method of claim 50 comprising:
forming an opening in the tendon or the ligament; and
threading the implant through the opening.

69. The method of claim 50 comprising locating more than 50% of the material comprising the implant in a center region between the first anchor portion and distal ends of the tension member.

70. The method of claim 50 comprising:
inserting a plurality of protrusions on a first layer of the first anchor portion through the tendon or ligament; and
engaging a second layer of the first anchor portion with the distal ends of the protrusions on the other side of the tendon or ligament.

71. The method of claim 50 comprising the step of:
engaging the tension member with tension adjusting device; and
adjusting tension on the tension member.
